(19) United States
(12) Patent Application Publication
(10) Pub. No.: US 2005/0288654 A1
(43) Pub. Date: Dec. 29, 2005

(54) Methods and devices for delivering ablative energy

(76) Inventors: Tim Nieman, North Salt Lake City, UT (US); Ninh Huu Dang, Trabuco Canyon, CA (US); Tazi Pruitt, Carlsbad, CA (US); Jim L. Ward, Laguna Niguel, CA (US); Erik Bulman, Mission Viejo, CA (US); Mathew R. Williams, New York, NY (US); John Murphy, North Reading, MA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/146,302

(22) Filed: Jun. 6, 2005

Related U.S. Application Data
(60) Provisional application No. 60/578,021, filed on Jun. 7, 2004.
(60) Provisional application No. 60/672,919, filed on Apr. 18, 2005.

(51) Int. Cl. A61B 18/20

(52) U.S. Cl. 606/13; 606/16; 606/17

(57) ABSTRACT

Ablation instruments and methods are disclosed for ablating diseased tissue such as cardiac tissue. The ablation device can remotely apply ablative energy to biological tissue and comprises a flexible elongate member having a proximal end, a distal end and a longitudinal lumen extending therebetween. An energy emitting element is disposed within the longitudinal lumen of the flexible elongate member. The energy emitting element has a proximal end and a distal end for emitting energy along at least a portion of its length. The device is configured to emit a variable amount of energy along a length of the flexible elongate member. The method includes introducing the flexible elongate member into a predetermined tissue site to ablate a target tissue. The target tissue is ablated, coagulated or photochemically modulated without damaging surrounding tissue.

### CROSS-REFERENCE TO RELATED APPLICATIONS

The pending application claims priority to U.S. Provisional Application No. 60/578,021 filed on Jun. 7, 2004 and to U.S. Provisional Application No. 60/672,919 filed on Apr. 18, 2005, which are hereby incorporated by reference in their entirety.

### FIELD OF THE INVENTION

The present invention relates to ablation devices for medical therapies. In particular, the present invention relates to ablation instrument systems that use energy to ablate internal bodily tissues, and methods for using such systems for the treatment of diseases. Even more particularly, the systems and methods of the present invention can be used, for example, in the treatment of cardiac conditions such as cardiac arrhythmias.

### BACKGROUND OF THE INVENTION

Cardiac arrhythmias, e.g., fibrillation, are irregularities in the normal beating pattern of the heart and can originate in either the atria or the ventricles. For example, atrial fibrillation is a form of arrhythmia characterized by rapid randomized contractions of the atrial myocardium, causing an irregular, often rapid ventricular rate. The regular pumping function of the atria is replaced by a disorganized, ineffective quivering as a result of chaotic conduction of electrical signals through the upper chambers of the heart. Atrial fibrillation is often associated with other forms of cardiovascular disease, including congestive heart failure, rheumatic heart disease, coronary artery disease, left ventricular hypertrophy, cardiomyopathy or hypertension.

Atrial arrhythmia may be treated using several methods. Pharmacological treatment of atrial fibrillation, for example, is initially the preferred approach, first to maintain normal sinus rhythm, or secondly to decrease the ventricular response rate. Other forms of treatment include drug therapies, electrical cardioversion, and RF catheter ablation of selected areas determined by mapping. In the more recent past, other surgical procedures have been developed for atrial fibrillation, including left atrial isolation, transvenous catheter or cryosurgical ablation of His bundle, and the Corridor procedure, which have effectively eliminated irregular ventricular rhythm. However, these procedures have for the most part failed to restore normal cardiac hemodynamics, or alleviate the patient's vulnerability to thromboembolism because the atria are allowed to continue to fibrillate. More effective surgical treatment was thus required to cure medically refractory atrial fibrillation of the heart.

Accordingly, more effective surgical techniques have been proposed to treat medically refractory atrial fibrillation of the heart. Although these procedures were originally performed with a scalpel, these techniques may also use ablation (also referred to as coagulation). One such technique is strategic ablation of the atrial tissues through ablation catheters that treat the tissue, generally with heat or cold, to cause tissue necrosis (i.e., cell destruction). The destroyed muscle cells are replaced with scar tissue which cannot conduct normal electrical activity within the heart.

For example, the pulmonary vein has been identified as one of the origins of errant electrical signals responsible for triggering atrial fibrillation. In one known approach, circumferential ablation of tissue within the pulmonary veins or at the ostia of such veins has been practiced to treat atrial fibrillation. Similarly, ablation of the region surrounding the pulmonary veins as a group has also been proposed. By ablating the heart tissue (typically in the form linear or curved lesions) at selected locations, electrical conductivity from one segment to another can be blocked and the resulting segments become too small to sustain the fibrillatory process on their own. Ablation procedures are often performed during coronary artery bypass and mitral valve replacement operations because of a heightened risk of arrhythmias in such patients and the opportunity that such surgery presents for direct access to the heart.

Several types of ablation devices have recently been proposed for creating lesions to treat cardiac arrhythmias, including devices which employ electrical current (e.g., radio-frequency “RF”) heating or cryogenic cooling. Such ablation devices have been proposed to create elongated lesions that extend through a sufficient thickness of the myocardium to block electrical conduction.

These devices, however, are not without their drawbacks. When cardiac surgery is performed “on pump,” the amount of time necessary to form a lesion becomes a critical factor. Because these devices rely upon resistive and conductive heating (or cooling), they must be placed in direct contact with the heart and such contact must be maintained for a considerable period of time to form a lesion that extends through the entire thickness of the heart muscle. The total length of time to form the necessary lesions can be excessive. This is particularly problematic for procedures that are performed upon a “beating heart” patient. In such cases the heart itself continues to beat and, hence, is filled with blood, thus providing a heat sink (or reservoir) that works against conductive and/or resistive ablation devices. As “beating heart” procedures become more commonplace (in order to avoid the problems associated with arresting a patient's heart and placing the patient on a pump), the need for better ablation devices will continue to grow.

Moreover, devices that rely upon resistive or conductive heat transfer can be prone to serious post-operative complications. In order to quickly perform an ablation with such “contact” devices, a significant amount of energy must be applied directly to the target tissue site. In order to achieve transmural penetration, the surface that is contacted will experience a greater degree of heating (or freezing). For example, in RF heating of the heart wall, a transmural lesion requires that the tissue temperature be raised to about 50° C. throughout the thickness of the wall. To achieve this, the contact surface will typically be raised to at least 80° C. Charring of the surface of the heart tissue can lead to the creation of blood clots on the surface which can lead to post-operative complications, including stroke. Even if structural damage is avoided, the extent of the lesion (i.e., the width of the ablated zone) on the surface that has been contacted will typically be greater than necessary.

Ablation devices that do not require direct contact have also been proposed, including acoustic and radiant energy. Acoustic energy (e.g., ultrasound) is poorly transmitted into tissue (unless a coupling fluid is interposed). Laser energy has also been proposed but only in the context of devices that focus light into spots or other patterns. When the light energy is delivered in the form of a focused spot, the process is inherently time consuming because of the need to expose numerous spots to form a continuous linear or curved lesion.

In addition, existing instruments for cardiac ablation also suffer from a variety of design limitations. The shape of the heart muscle adds to the difficulty in accessing cardiac structures, such as the pulmonary veins which are located on the posterior surface of the heart. Further, the presence of epicardial fat limits the depth of ablative penetration for many ablative energy sources.

Accordingly, there exists a need for better surgical ablation instruments that can form lesions with minimal overheating and/or damage to collateral tissue. Moreover, instruments that are capable of creating lesions uniformly, rapidly and efficiently would satisfy a significant need in the art.

### SUMMARY OF THE INVENTION

The present invention provides surgical ablation instrument systems for creating lesions in tissue, especially cardiac tissue for treatment of arrhythmias and other cardiac conditions. The hand held instruments are especially useful in open chest or port access cardiac surgery for rapid and efficient creation of curvilinear lesions to serve as conduction blocks. The instruments can be applied to form either endocardial or epicardial ablations, and are designed to create lesions in the atrial tissue in order to electrically decouple tissue segments on opposite sides of the lesion.

In one aspect of the invention, surgical ablation instruments are disclosed that are well adapted for use in or around the intricate structures of the heart. In one embodiment, the distal end of the instrument can have a malleable shape so as to conform to the surgical space in which the instrument is used. The instruments can include at least one malleable strip element disposed within the distal end of the instrument body or housing so that the distal end can be conformed into a desired shape. In addition, the instruments can also include a clasp to form a closed loop after encircling a target site, such as the pulmonary veins. Such instruments can be used not only with penetrating energy devices but also with other ablation means, such as RF heating, cryogenic cooling, ultrasound, microwave, ablative fluid injection and the like. In still another embodiment, the distal end of the instrument can include a translatory mechanism for disposing the tip of the instrument in a variety of configurations.

In one embodiment, the surgical ablation instrument includes a housing or flexible elongate member having a proximal end, a distal end and a longitudinal lumen extending therebetween. An energy emitting element having a proximal end and a distal end can be slidably disposed within the lumen for transmitting energy to the distal end of the elongate member. The housing can comprise a plurality of interconnected links, or can include cutout portions such as grooves on its outer surface to facilitate flexion. The housing can also be formed from a flexible strip or flexible bellows.

In another aspect of the invention, the housing can include a profile that provides for longitudinal flexibility as well as torsional strength. In one embodiment, the housing includes a shaped inner lumen for containing a complementarily shaped light delivering element. The specific geometries of the lumen and element are such that twisting or rotation of the light delivering element within the inner lumen is prevented, and the orientation of the light delivering element with respect to the housing is ensured. In another embodiment, the housing can include reinforcement such as shape memory wire or polymeric supports to prevent the housing from twisting when positioned on tortuous anatomical surfaces.

In one aspect of the invention, hand-held and percutaneous instruments are disclosed that can achieve rapid and effective photoablation through the use of penetrating radiation, especially distributed radiant energy. It has been discovered that radiant energy, e.g., diffuse infrared radiation, can create lesions in less time and with less risk of the adverse types of tissue destruction commonly associated with prior art approaches. Unlike instruments that rely on thermal conduction or resistive heating, controlled penetrating radiant energy can be used to simultaneously deposit energy throughout the full thickness of a target tissue, such as a heart wall, even when the heart is filled with blood. Distributed radiant energy can also produce better defined and more uniform lesions.

It has also been discovered that infrared radiation is particularly useful in forming photoablative lesions. In one preferred embodiment the instruments emit radiation at a wavelength in a range from about 800 nm to about 1000 nm, and preferably emit at a wavelength in a range of about 915 nm to about 980 nm. Radiation at a wavelength of 915 nm or 980 nm is commonly preferred, in some applications, because of the optimal absorption of infrared radiation by cardiac tissue at these wavelengths. In the case of ablative radiation that is directed towards the epicardial surface, light at a wavelength about 915 nm can be particularly preferably.

In another aspect of the invention, surgical ablation instruments are disclosed that are well adapted for use in or around the intricate structures of the heart. In one embodiment, the distal end of the instrument can have a malleable shape so as to conform to the surgical space in which the instrument is used. Optionally, the distal end of the instrument can be shaped into a curve having a radius between about 5 millimeters and about 25 millimeters. The instruments can include at least one malleable strip element disposed within the distal end of the instrument body or housing so that the distal end can be conformed into a desired shape. In addition, the instruments can also include a clasp to form a closed loop after encircling a target site, such as the pulmonary veins.

In yet another aspect of the invention, surgical ablation instruments are disclosed having a housing with at least one lumen therein and having a distal portion that is at least partially transmissive to photoablative radiation. The instruments further include a light delivery element within the lumen of the housing that is adapted to receive radiation from a source and deliver radiant energy through a transmissive region of the housing to a target tissue site. The radiant energy is delivered without the need for contact between the light emitting element and the target tissue because the instruments of the present invention do not rely upon conductive or resistive heating.

In other aspects of the invention, ablation instruments are provided having a sufficient length to create a full encircling path around the pulmonary veins. The instruments can be configured to emit varying amounts of ablative energy along its length. In one embodiment, the ablation device includes an energy emitting element that comprises a plurality of segments, each segment having a different diameter than an adjacent segment to collectively form an elongate energy emitting element having variable diameters along its length. The energy emitting element can also be provided with a tapered profile along its length, in order to vary the amount of ablative energy emitted. The instrument can be used to provide an ablative path around both pairs of pulmonary veins, or an individual pair of pulmonary veins.

In another embodiment, the instrument can include an inflatable elongate balloon that resides within the housing along with the light delivering element. An inflation controller in communication with the balloon and an inflation source, e.g., an air, gas or fluid pump, can be provided to enable the selective inflation of the balloon. Upon inflation, the balloon urges against the light delivering element and effects the angular orientation of the element with respect to the longitudinal axis of the housing. This allows the surgeon to change the angle of the light delivering element by controlling the inflation of the balloon, and consequently the energy emitting pathway along the length of the light delivering element.

In yet another embodiment, the instrument can include a plurality of light delivering elements of varying lengths, each element being configured to emit a dose of ablative energy at a specific position with respect to the length of the housing. Each of the light delivering elements can have a different length than the other elements. A selection mechanism can be provided with the ablation instrument so that the surgeon can select any one of the plurality of light delivering elements for activation. Preferably, each of the light delivering elements includes a diffuser tip at a distal end. The instrument can include a housing that has a portion transparent to emitted energy.

The light delivering element can be a light transmitting optical fiber adapted to receive ablative radiation from a radiation source and a light emitting tip at a distal end of the fiber for emitting diffuse or defocused radiation. The light delivering element can be slidably disposed within the inner lumen of the housing and the instrument can further include a translatory mechanism for disposing the tip of the light delivering element at one or more of a plurality of locations with the housing. Optionally, a lubricating fluid can be disposable between the light delivery element and the housing. This fluid can be a physiologically compatible fluid, such as saline, and the fluid can also be used for cooling the light emitting element or for irrigation via one or more exit ports in the housing.

In one embodiment of the invention, the ablation device comprises a housing having a proximal end, a distal end and a longitudinal lumen extending therebetween. An ablation element is disposed within the lumen of the housing to ablate tissue at a target site. Also included is an irrigation cap at the distal end of the ablation element. A fluid source connected to the housing provides fluid to the ablation element during delivery of the ablation energy. The fluid can be introduced via a fluid inlet on the irrigation cap to be delivered between the ablation element and the irrigation cap. A cutout portion formed within the irrigation cap forms a fluid carrying cavity for delivering the fluid to the ablation element. In one particular aspect, the irrigation cap is formed as a pair of jaws, with the free ends of the jaws having surface features such as teeth, grooves, etc. for enhanced gripping. The fluid can comprise a material which cools the ablation element during delivery of ablative energy, and can include lubricating fluids, and/or physiologically compatible fluids such as saline.

The light emitting tip can include a hollow tube having a proximal end joined to the light transmitting optical fiber, a closed distal end, and an inner space defining a chamber therebetween. The light scattering medium disposed within the chamber can be a polymeric or liquid material having light scattering particles, such as alumina, silica, or titania compounds or mixtures thereof, incorporated therein. The distal end of the tube can include a reflective end and, optionally, the scattering medium and the reflective end can interact to provide a substantially uniform axial distribution of radiation over the length of the housing.

Alternatively, the light emitting tip can include at least one reflector for directing the radiation through the transmissive region of the housing toward a target site and, optionally can further include a plurality of reflectors and/or at least one defocusing lens for distributing the radiation in an elongated pattern.

The light emitting tip can further include at least one longitudinal reflector or similar optical element such that the radiation distributed by the tip is confined to a desired angular distribution. In one embodiment, the reflector is configured to selectively block a portion of the energy emitting element from emitting ablative energy. The reflector can be configured to seat around the energy emitting element, and can include a window or cutout portion for emitting energy. The window can be adjustably positioned along the length of the reflector. Alternatively, or in addition, the size of the window can also be adjustable.

The hand held instruments can include a handle incorporated into the housing. An inner lumen can extend through the handle to received the light delivering element. The distal end of the instrument can be resiliently deformable or malleable to allow the shape of the ablation element to be adjusted based on the intended use.

In one embodiment, a hand held cardiac ablation instrument is provided having a housing with a curved shape and at least one lumen therein. A light delivering element is disposable within the lumen of the housing for delivering ablative radiation to form a curved lesion at a target tissue site adjacent to the housing.

In another aspect of the invention, the light delivering element can be slidably disposed within the inner lumen of the housing, and can include a light transmitting optical fiber adapted to receive ablative radiation from a radiation source and a light diffusing tip at a distal end of the fiber for emitting radiation. The instrument can optionally include a handle joined to the housing and having an inner lumen though which the light delivering element can pass from the radiation source to the housing.

In yet another aspect of the present invention, the light diffusing tip can include a tube having a proximal end mated to the light transmitting optical fiber, a closed distal end, and an inner chamber defined therebetween. A light scattering medium is disposed within the inner chamber of the tube. The distal end of the tube can include a reflective end surface, such as a mirror or gold coated surface. The tube can also include a curved, longitudinally-extending, reflector that directs the radiant energy towards the target ablation site. The reflective surfaces and the light scattering medium interact to provide a substantially uniform axial distribution of radiation of the length of the housing.

In other aspects of the present invention, a hand held cardiac ablation instrument is provided having a slidably disposed light transmitting optical fiber, a housing in the shape of an open loop and having a first end adapted to receive the slidably disposed light transmitting optical fiber, and at least one diffuser chamber coupled to the fiber and disposed within the housing. The diffuser chamber can include a light scattering medium disposed within the housing and coupled to the slidably disposed light transmitting optical fiber.

In yet another aspect, a percutaneous cardiac ablation instrument in the form of a balloon catheter with an ablative light projecting assembly is provided. The balloon catheter instrument can include at least one expandable membrane disposed about a housing. This membrane is generally or substantially sealed and serves as a balloon to position the device within a lumen. The balloon structure, when filled with fluid, expands and is engaged in contact with the tissue. The expanded balloon thus defines a staging from which to project ablative radiation in accordance with the invention. The instrument can also include an irrigation mechanism for delivery of fluid at the treatment site. In one embodiment, irrigation is provided by a sheath, partially disposed about the occluding inner balloon, and provides irrigation at a treatment site (e.g. so that blood can be cleared from an ablation site). The entire structure can be deflated by applying a vacuum which removes the fluid from the inner balloon. Once fully deflated, the housing can be easily removed from the body lumen.

The present invention also provides methods for ablating tissue. One method of ablating tissue comprises positioning a distal end of a penetrating energy instrument in proximity to a target region of tissue, the instrument including a source of penetrating energy disposed within the distal end. The distal end of the instrument can be curved to permit the distribution of penetrating energy in elongated and/or arcuate patterns. The method further including activating the energy element to transmit penetrating energy to expose the target region and induce a lesion; and, optionally, repeating the steps of positioning and exposing until a composite lesion of a desired shape is formed.

In another method, a device is provided having a light delivering element coupled to a source of photoablative radiation and configured in a curved shape to emit an arcuate pattern of radiation. The device is positioned in proximity to a target region of cardiac tissue, and applied to induce a curvilinear lesion. The device is then moved to the second position and reapplied to induce a second curvilinear lesion. The steps of positioning and reapplying can be repeated until the lesions are joined together to create a composite lesion (e.g., a closed loop encircling one or more cardiac structures).

In another embodiment, methods of ablating cardiac tissue are provided. A device is provided having a housing in the shape of a hollow ring or partial ring having at least one lumen therein and at least one open end, and a light delivering element slidably disposed within the lumen of the housing for delivering ablative radiation to form a circular lesion at a target region adjacent the housing. The methods includes the steps of positioning the device in proximity to the target region of cardiac tissue, applying the device to the target region to induce a curvilinear lesion, advancing the light delivering element to a second position, reapplying the device to the target region to induce a second curvilinear lesion, and repeating the steps of advancing and applying until the lesions are joined together to create a composite circumferential lesion.

### BRIEF DESCRIPTION OF THE DRAWINGS

The invention will be more fully understood from the following detailed description taken in conjunction with the accompanying drawings, in which like reference numerals designate like parts throughout the figures, and wherein:

FIG. 1 is a schematic, perspective view of a hand held surgical ablation instrument in accordance with this invention;

FIG. 1A is a partially cross-sectional view of the hand held surgical ablation instrument of FIG. 1;

FIG. 1B is a perspective view of the handle and light delivering element of the hand held surgical ablation instrument of FIG. 1A;

FIG. 2 is a schematic, perspective view of another embodiment of a hand held surgical ablation instrument in accordance with this invention;

FIG. 2A is a partially cross-sectional view of the hand held surgical ablation instrument of FIG. 2;

FIG. 3 is a schematic, side perspective view of a tip portion of an ablation instrument in accordance with another embodiment of the invention illustrating a light delivery element;

FIG. 3A is a schematic, side perspective view of a tip portion of another ablation instrument in accordance with the invention;

FIG. 4 is a schematic, cross sectional view of the light delivery element of FIG. 3;

FIG. 4A is a schematic, cross sectional view of another embodiment of a light delivery element;

FIG. 4B is a schematic, cross sectional view along the length of an irrigation cap and light delivery element of another embodiment the present invention;

FIG. 4C is a schematic, cross sectional side view of the irrigation cap and light delivery element of FIG. 4B;

FIG. 5 is a schematic, cross sectional view of another embodiment of a light delivery element surrounded by a malleable housing;

FIG. 6 is a perspective view of another embodiment of a flexible housing;

FIG. 6A is an enlarged, perspective view of the flexible housing of FIG. 6;

FIG. 6B is an exploded view of the flexible housing of FIG. 6;

FIG. 7A is a schematic, cross sectional view of another embodiment of an ablation element of the present invention;

FIG. 7B is a schematic, cross sectional view of another embodiment of an ablation element of the present invention;

FIG. 7C is a schematic, cross sectional view of another embodiment of an ablation element of the present invention;

FIG. 7D is a schematic, cross sectional view of another embodiment of an ablation element of the present invention;

FIG. 7E is a schematic, cross sectional view of another embodiment of an ablation element of the present invention;

FIG. 7F is a schematic, cross sectional view of another embodiment of an ablation element of the present invention;

FIG. 8 illustrates an ablation element in position around the pulmonary veins of a heart;

FIG. 8A is a perspective side view of one embodiment of the ablation element of FIG. 8;

FIG. 8B is a perspective cross sectional view of a reflector of the ablation element of FIG. 8A;

FIG. 8C is a perspective side view of another embodiment of the ablation element of FIG. 8;

FIG. 8D is a perspective cross sectional view of a reflector of the ablation element of FIG. 8C;

FIG. 8E is a perspective side view of yet another embodiment of the ablation element of FIG. 8;

FIG. 8F is a perspective cross sectional view of a reflector of the ablation element of FIG. 8E;

FIG. 8G is a perspective side view of even still another embodiment of the ablation element of FIG. 8;

FIG. 8H is a perspective cross sectional view of a reflector of the ablation element of FIG. 8G;

FIG. 9 is a perspective view of another embodiment of an ablation element of the present invention;

FIG. 10 is a schematic, cross sectional top view of a surgical ablation element of according to the invention, illustrating the different ablating positions of the light delivering element;

FIG. 11 is a schematic, perspective view of a human heart and an instrument according to the invention, showing one technique for creating epicardial lesions;

FIG. 12 is a schematic, perspective view of a human heart and an instrument according to the invention, showing one technique for creating endocardial lesions;

FIG. 13 is a schematic, perspective view of a human heart and an instrument according to the invention, showing another technique for creating endocardial lesions;

FIG. 14A is a perspective cross sectional view of yet another embodiment of an ablation element of the present invention;

FIG. 14B is a perspective cross sectional view still yet another embodiment of an ablation element of the present invention;

FIG. 14C is a perspective cross sectional view of another embodiment of an ablation element of the present invention;

FIG. 14D is a perspective cross sectional view of still another embodiment of an ablation element of the present invention;

FIG. 15 is an exploded schematic view of another embodiment of an ablation instrument of the present invention;

FIG. 16 is a schematic, perspective view of a human heart and an instrument according to the invention, showing yet another technique for creating endocardial lesions;

FIG. 17A is a perspective view of a flexible guidewire of the present invention;

FIG. 17B is a perspective side view of an ablation instrument of the present invention;

FIG. 17C is another perspective side view of the ablation instrument of FIG. 17B;

FIG. 17D is yet another a perspective side view of the ablation instrument of 17B;

FIG. 18A is a perspective view of a flexible guidewire of the present invention;

FIG. 18B is a perspective side view of an ablation instrument of the present invention;

FIG. 18C is another perspective side view of the ablation instrument of FIG. 18B;

FIG. 18D is yet another a perspective side view of the ablation instrument of 18B;

FIG. 19 is a perspective view of yet another embodiment of a cardiac ablation instrument of the present invention;

FIG. 19A is a cross-sectional view of the ablation instrument of FIG. 19;

FIG. 19B is an exploded view of the ablation instrument of FIG. 19;

FIG. 20A is an exploded view of the guide or tip of the instrument of FIG. 19;

FIG. 20B is a perspective exterior view of the guide or tip of the instrument of FIG. 19;

FIG. 20C is a perspective cross-sectional view of the guide or tip of FIG. 20B;

FIG. 21A is an exploded view of the extension to sheath assembly of FIG. 19;

FIG. 21B is a cross-sectional view of the extension to sheath assembly of FIG. 19;

FIG. 22 is an exploded view of the handle portion of FIG. 19;

FIG. 22A is an enlarged detailed view of the indexing button of FIG. 22;

FIG. 22B is a cross-sectional view of the handle portion of FIG. 22; and

FIG. 23 is cross-sectional view of another embodiment of the ablation instrument shown in FIG. 19.

### DETAILED DESCRIPTION OF THE INVENTION

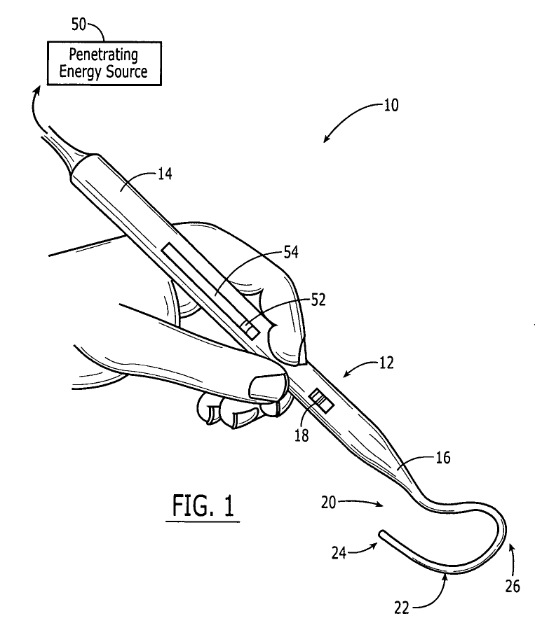

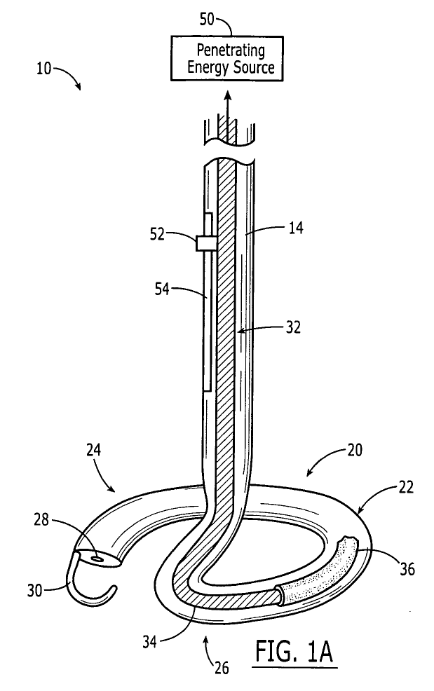

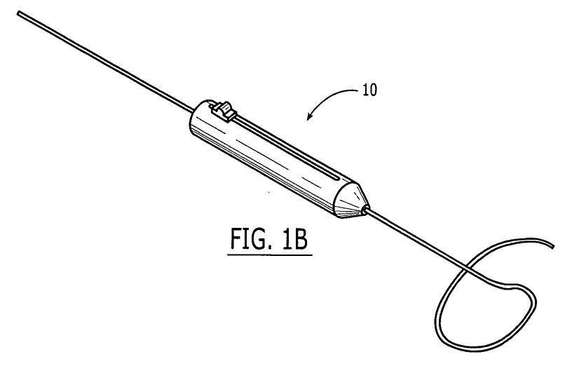

The present invention provides hand held surgical ablation instruments that are useful for treating patients with cardiac conditions such as, for example, atrial arrhythmia. Turning now to the drawings and particularly to FIG. 1, an exemplary embodiment of a hand held cardiac ablation instrument 10 in accordance with the present invention is shown. Ablation instrument 10 generally includes a handle 12 having a proximal end 14 and a distal end 16, an ablation element 20 mated to or extending distally from the distal end 16 of the handle 12, and a penetrating energy source 50. The energy source 50 can be, for example, a laser source of radiation, e.g., coherent light, which can be efficiently and uniformly distributed to the target site while avoiding harm or damage to surrounding tissue. In use, the instrument 10 can be applied either endocardially or epicardially, and is effective to uniformly irradiate a target ablation site.

The handle 12 of the ablation instrument 10 is effective for manually placing the ablation element 20 proximate to a target tissue site. While the handle 12 can have a variety of shapes and sizes, preferably the handle 12 is generally elongate with at least one inner lumen extending therethrough. The proximal end 14 of the handle 12 can be adapted for coupling with a source of radiant energy 50, and the distal end of the handle 16 is mated to or formed integrally with the ablation element 20. In a preferred embodiment, the handle 12 is positioned substantially coaxial with the center of the ablation element 20. The handle 14 can optionally include an on-off switch 18 for activating the laser energy source 50.

As shown in more detail in FIG. 1A, the ablation element 20 can include an outer housing 22 having an inner lumen extending therethrough, and a light delivering element 32 disposed within the inner lumen of the outer housing 22. The outer housing 22 can be flexible, and is preferably malleable to allow the shape of the outer housing 22 to conform to various anatomical shapes as needed. The light delivering element 32 which is disposed within the outer housing 22 includes a light transmitting optical fiber 34 and a light diffusing tip 36. The light transmitting optical fiber 34 is adapted to receive ablative energy from a penetrating energy source 50 and is effective for delivering radiant energy from the laser energy source 50 to the light diffusing tip 36, wherein the laser energy is diffused throughout the tip 36 and delivered to the target ablation site.

The light delivering element 32 can be slidably disposed within the outer housing 22 to allow the light diffusing tip 36 to be positioned with respect to the target ablation site. A lever 52 or similar translatory mechanism can be provided for slidably moving the light delivering element 32 with respect to the handle 12. As shown in FIGS. 1A and 1B (which shows the handle 12 with the light delivering element 32 slidably contained therein without the outer housing 22), the lever 52 can be mated to the light delivering element 32 and can protrude from a distally extending slot 54 formed in the handle 12. In this configuration, translatory movement of the lever 52 effects advancement or sliding of the light delivering element 32 to selectively place the light delivering element 32 at a discrete position within the outer housing 22 and proximate to the tissue surface to be ablated. Markings can also be provided on the handle 12 for determining the distance moved and the length of the lesion formed. A person skilled in the art will readily appreciate that a variety of different mechanisms can be employed to slidably move the light delivering element 32 with respect to the handle 12.

The outer housing 22 can optionally include a connecting element for forming a closed-loop circumferential ablation element 20. By non-limiting example, FIG. 1A illustrates a connecting element 30 extending from the leading, distal end 24 of the outer housing 22. The connecting element 30 has a substantially u-shape and is adapted for mating with the trailing end 26 of the outer housing 22 or the distal end 16 of the handle 12. The connecting element 30 can optionally be adapted to allow the size of the circumferential ablation element 20 to be adjusted once positioned around the pulmonary veins. For example, the connecting element 30 can be positioned around the trailing end 26 of the outer housing 22 after the circumferential ablation element 20 is looped around the pulmonary veins, and the handle 12 can then be pulled to cause the ablation element 20 to tighten around the pulmonary veins. While FIG. 1A illustrates a U-shaped connecting element, a person having ordinary skill in the art will appreciate that a variety of different connecting elements or clasps 30 can be used such as, for example, a hook, a cord, a snap, or other similar connecting device.

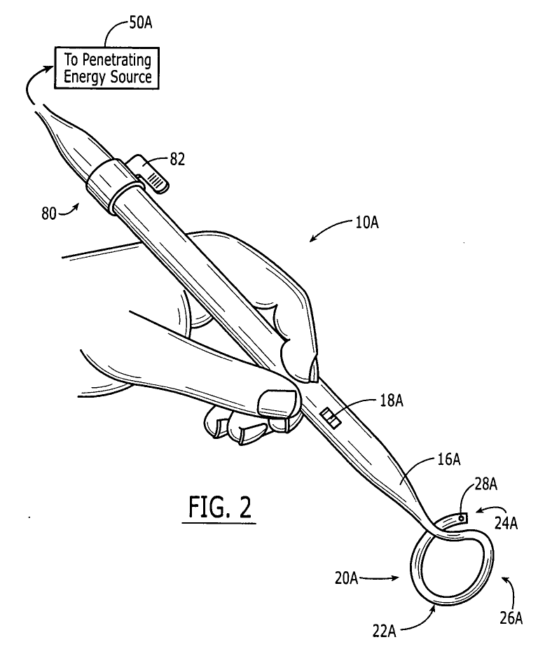

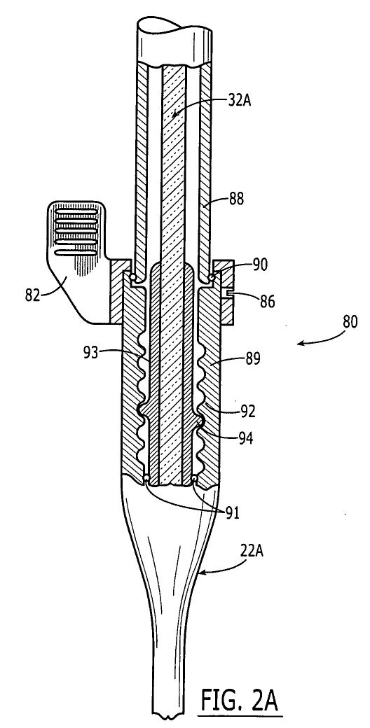

Another embodiment of the surgical ablation instrument 10A is shown in FIG. 2, where a rotatable lever 82 can be used to control the positioning of a light delivery element in the distal tip of the instrument. The lever 82 turns a translatory mechanism 80, as shown in more detail in FIG. 2A. In this embodiment, a portion 84 of the handle is separated from the rest of the housing 88 such that it can rotate, and preferably sealed by O-rings 90 and 91, or the like. The rotatable segment 84 has internal screw threads 92. Within this segment of the handle, the light delivering fiber 32A is joined to a jacket 93 that has an external screw thread 94. The threads 94 of jacket 93 mate with the threads 92 of rotatable segment 84. The lever 82 is affixed to rotatable segment 84 (e.g., by set screw 86) such that rotation of knob 82 causes longitudinal movement of the fiber 32A relative to the housing 88.

The outer housing 22A can be preshaped to function as a guide device to guide the light delivering element 32A along the ablation path. The cooperation between the light delivering element 32A and the inner lumen, as the element 32A is advanced through the inner lumen, positions the ablative element in a proper orientation to facilitate ablation of the targeted tissue during the advancement. Thus, once the outer housing 22A is stationed relative to the targeted tissue site, the light delivering element 32A can be easily advanced along the ablation path to generate the desired tissue ablations.

As shown in FIG. 2, the outer housing 22A can be in the shape of a hollow ring (or partial ring) forming an opening loop having leading and trailing ends 24A, 26A. The open loop-shape allows the circumferential ablation element 20A to be positioned around one or more pulmonary veins. While an open loop shape is illustrated, the outer housing 22A can also be formed or positioned to create linear or other shaped lesions. The slidable passing of the light delivering element can be performed by incrementally advancing the light delivering element 32A along a plurality of positions on the ablation path to produce a substantially continuous lesion.

The inner lumen of the outer housing 22, 22A in FIGS. 1 and 2 can optionally contain a lubricating or irrigating fluid to assist the light delivering element 32, 32A as it is slidably moved within the outer housing 22, 22A. The fluid can also cool the light delivering element 32, 32A during delivery of ablative energy. Fluid can be introduced using techniques known in the art, but is preferably introduced through a port and lumen formed in the handle. The distal end 24, 24A of the outer housing 22, 22A can include a fluid outflow port 28, 28A for allowing fluid to flow therethrough.

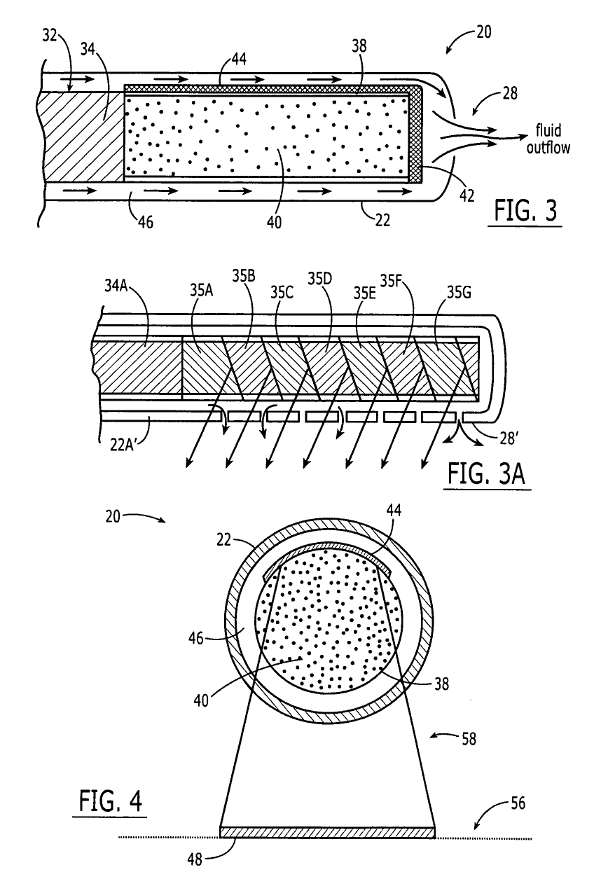

As shown in FIG. 3, which illustrates a portion of ablation instrument 10, the fluid travels between the light delivering element 32 toward the leading, distal end 26 of the outer housing 22 and exits the fluid outflow port 28. Since the port 28 is positioned on the distal end 26 of the outer housing 22, the fluid does not interfere with the ablation procedure. While FIG. 3 illustrates the fluid outflow port 28 disposed on the distal end 24 of the outer housing 22, a person skilled in the art will readily appreciate that the fluid outflow port 28 can be disposed anywhere along the length of the outer housing 22.

In FIG. 3A another embodiment of a light delivery element according to the invention is shown in which fiber 34A terminates in a series of partially reflective elements 35A-35G. A person skilled in the art should be appreciated that the number of reflective elements can vary depending on the application and the choice of six is merely for illustration. The transmissivity of the various segments can be controlled such that, for example, segment 35A is less reflective than segment 35B, which in turn is less reflective than 35C, etc., in order to achieve uniform diffusion of the light. The reflective elements of FIG. 3A can also be replaced, or augmented, by a series of light scattering elements having similar progressive properties. FIG. 3A also illustrates another arrangement of exit ports 28′ in housing 22A′ for fluid release, whereby the fluid can be used to irrigate the target site.

With reference again to FIG. 3, the light transmitting optical fiber 34 generally includes an optically transmissive core surrounded by a cladding and a buffer coating (not shown). The optical fiber 34 should be flexible to allow the fiber 34 to be slidably moved with respect to the handle 12. In use, the light transmitting optical fiber 34 conducts light energy in the form of ultraviolet light, infrared radiation, or coherent light, e.g., laser light. The fiber 34 can be formed from glass, quartz, polymeric materials, or other similar materials which conduct light energy.

The light diffusing tip 36 extends distally from the optical fiber 34 and is formed from a transmissive tube 38 having a light scattering medium 40 disposed therein. For additional details on construction of light diffusing elements, see, for example, U.S. Pat. No. 5,908,415 issued Jun. 1, 1999.

The scattering medium 40 disposed within the light diffusing tip 36 can be formed from a variety of materials, and preferably includes light scattering particles. The refractive index of the scattering medium 40 is preferably greater than the refractive index of the housing 22. In use, light propagating through the optical fiber 34 is transmitted through the light diffusing tip 36 into the scattering medium 40. The light is scattered in a cylindrical pattern along the length of the light diffusing tip 36 and, each time the light encounters a scattering particle, it is deflected. At some point, the net deflection exceeds the critical angle for internal reflection at the interface between the housing 22 and the scattering medium 40, and the light exits the housing 22 to ablate the tissue.

Preferred scattering medium 40 includes polymeric material, such as silicone, epoxy, or other suitable liquids. The light scattering particles can be formed from, for example, alumina, silica, or titania compounds, or mixtures thereof. Preferably, the light diffusing tip 36 is completely filled with the scattering medium 40 to avoid entrapment of air bubbles.

As shown in more detail in FIG. 3, the light diffusing tip 36 can optionally include a reflective end 42 and/or a reflective coating 44 extending along a length of one side of the light diffusing tip 36 such that the coating is substantially diametrically opposed to the target ablation site. The reflective end 42 and the reflective coating 44 interact to provide a substantially uniform distribution of light throughout the light diffusing tip 36. The reflective end 42 and the reflective coating 44 can be formed from, for example, a mirror or gold coated surface. While FIG. 3 illustrates the coating extending along one side of the length of the diffusing tip 36, a person having ordinary skill in the art will appreciate that the light diffusing tip 36 can be coated at different locations relative to the target ablation site. For example, the reflective coating 44 can be applied over 50% of the entire diameter of the light diffusing tip 36 to concentrate the reflected light toward a particular target tissue site, thereby forming a lesion having a relatively narrow width.

In one use, the hand held ablation instrument 10 is coupled to a source of penetrating energy 50 and can be positioned within a patient's body either endocardially or epicardially to ablate cardiac tissue. When the penetrating energy is light, the source is activated to transmit light through the optical fiber 34 to the light diffusing tip 36, wherein the light is scattered in a circular pattern along the length of the tip 36. The tube 38 and the reflective end 42 interact to provide a substantially uniform distribution of light throughout the tip 36. When a mirrored end cap 42 is employed, light propagating through the light diffusing tip 36 will be at least partially scattered before it reaches the mirror 42. When the light reaches the mirror 42, it is then reflected by the mirror 42 and returned through the tip 36. During the second pass, the remaining radiation encounters the scattering medium 40 which provides further diffusion of the light.

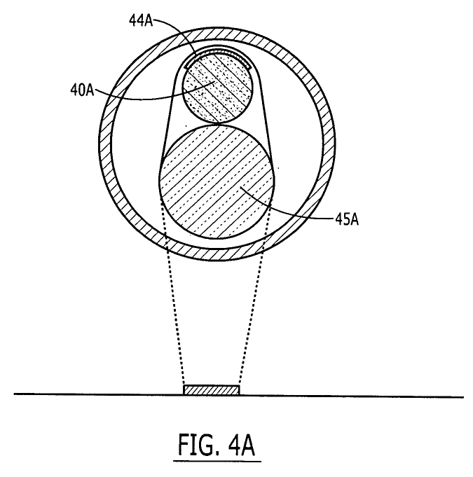

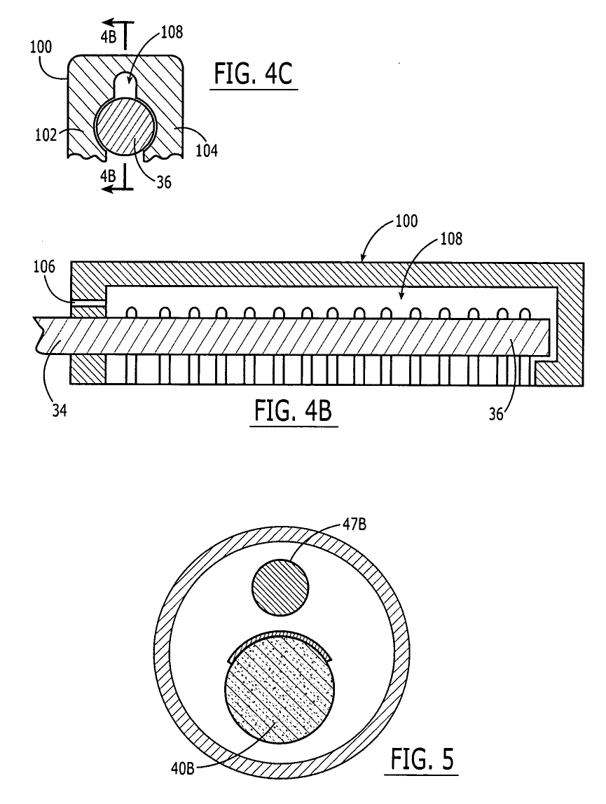

When a reflective coating or longitudinally disposed reflector 44 is used, as illustrated in FIG. 4, the light 58 emitted by the diffusing tip 36 will reflected toward the target ablation site 56 to ensure that a uniform lesion 48 is created. The reflective coating or element 44 is particularly effective to focus or direct the light 58 toward the target ablation site 56, thereby preventing the light 58 from passing through the housing 22 around the entire circumference of the housing 22.

In another embodiment as illustrated in FIG. 4A, the light emitting element can further include a longitudinally extended lens element 45A, such that light scattered by the scattering medium 40A is not only reflected by reflector 44A but also confined to a narrow angle.

In another aspect of the present invention, an irrigation cap 100 can be placed over the diffusing tip 36, as illustrated in FIG. 4B. The irrigation cap 100 can be formed from a flexible material such as silicone. The irrigation cap 100 includes a pair of attached jaws 102, 104. As shown in cross-section in FIG. 4C, the interior of the irrigation cap 100 includes a shaped cutout portion that is configured to fit over the optical fiber 34 like an open bracket that surrounds a portion of the optical fiber 34. The irrigation cap 100 also includes a fluid inlet 106 for the introduction of an irrigation or lubricating fluid between the light delivering element 32 and the cap 100. When the optical fiber 36 and diffusing tip 36 are captured within the cutout portion as shown in FIGS. 4B and 4C, a fluid carrying cavity 108 is formed as part of the cutaway portion of the cap 100. In use, fluid enters through the inlet 102 and into the cavity 108 where it cools the optical fiber 34. The excess fluid flows around the crevices between the optical fiber 34 and the irrigation cap 100, exiting from the cap 100 in the space between the jaws 102, 104. Preferably, the free ends of the jaws 102, 104 include surface features 110 such as grooves or teeth to provide for better gripping.

In yet another embodiment of the invention, illustrated in FIG. 5, the housing that surrounds the light delivery element 40B can include or surround a malleable element 47B, e.g., a soft metal bar or strip such that the clinician can form the distal end of the instrument into a desired shape prior to use. Although the malleable element 47B is shown embedded in the housing, it should be clear that it can also be incorporated into the light delivery element (e.g., as part of the longitudinally extended reflector) or be distinct from both the housing and the light emitter.

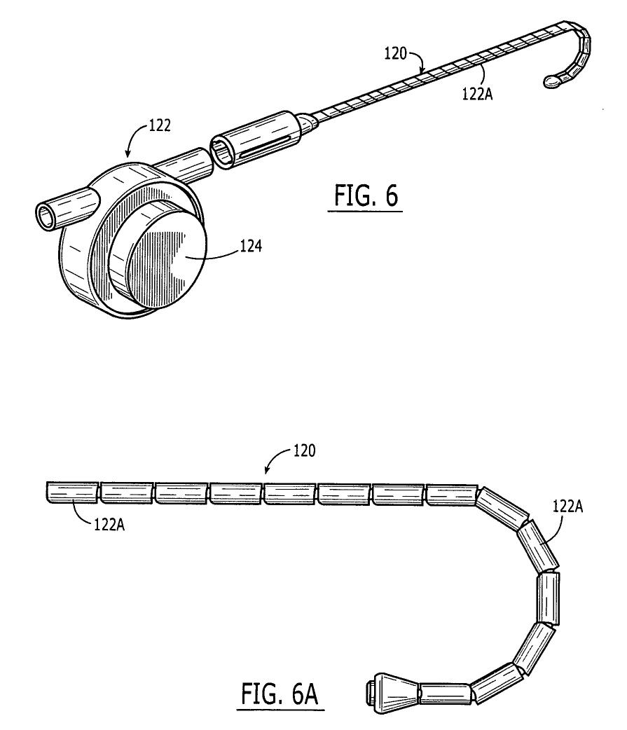

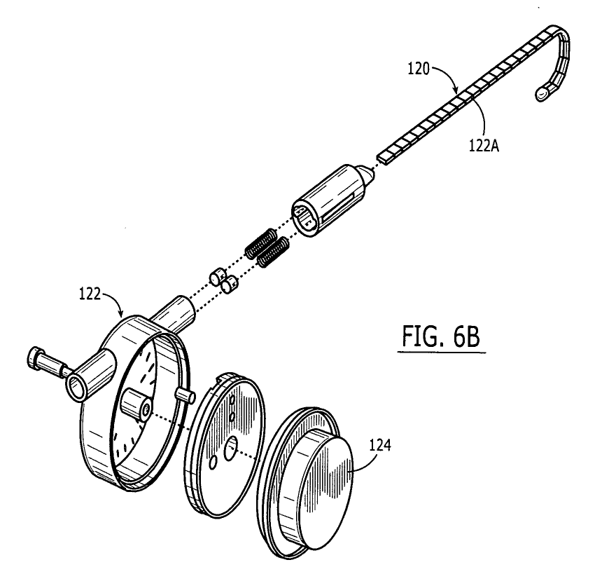

In still yet another embodiment of the invention, the outer housing 122A can comprise a plurality of linked units 120, as shown in FIGS. 6 and 6A, with FIG. 6B representing an exploded view of the outer housing. The linked units can be flexibly interconnected so that the housing 122A can bend into a desired shape. The housing 122A is associated with a control mechanism 122 that effects the movement of the units 120. For instance, a rotatable knob 124 can be implemented for bending the distal end of the outer housing 122A. The rotatable knob 124 can be associated with a wire or elongated filament (not shown) attached to the distal end of the housing 122A such that, upon rotation of the knob 124, the wire or filament is moved distally or proximally to cause longitudinal movement of the wire relative to the housing 122A. Preferably, the wire is a shape memory wire having a preformed shape such that the outer housing 122A can take the preshaped form.

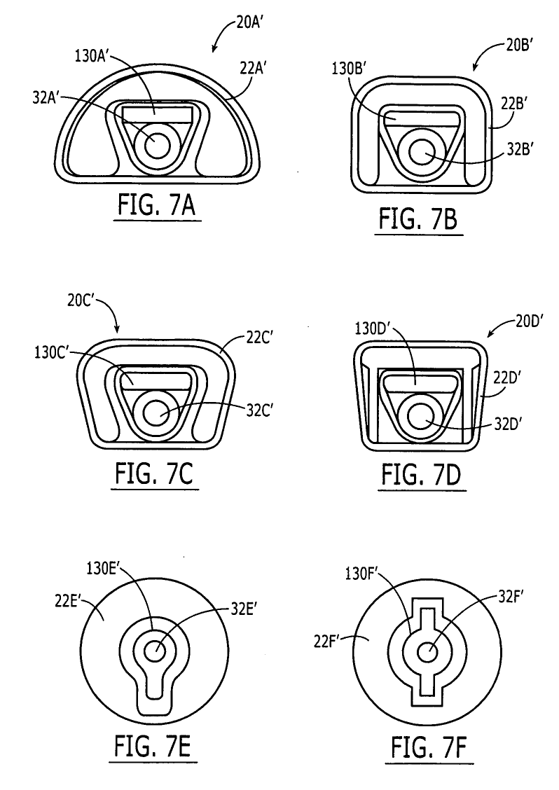

In another aspect of the invention, the ablation element, including the housing and inner lumen, can be configured with a special geometry to align the light delivering element and the outer housing. As illustrated in FIGS. 7A-7F, the outer housing 22A′-22F′ and the inner lumen of the instrument 20A′-20F′ can have a variety of shapes, while the light delivering element 32A′-32F′ can also have a special geometry that is complementary to the shape of the inner lumen of the outer housing 22A′-22F′. For instance, the light delivering element 32A′-32F′ can include a shape creating element 130A′-130F′ to ensure that the light delivering element 32A′-32F′ is aligned with the inner lumen of the outer housing 22A′-22F′. For instance, as illustrated in FIGS. 7A-7D, the light delivering element 32A′-32D′ can be heat shrunk around the shape creating element 130A′-130D′ to form a unique, pyramidal profile that limits the orientation and direction of the emitted ablation energy. With this profile, the light delivering element 32A′-32D′ is prevented from rotating within the housing 22A′-22D′ as it is sliding. The shape creating element 130A′-130D′ can be, for example, a shape memory flat wire (e.g., Nitinol flat wire) as illustrated, a polymer ribbon, or any protruding device that can be adhered to or incorporated with the light delivering element 32A′-32D′ to create a unique profile complementary to the inner lumen of the housing 22A′-22D′. FIGS. 7E and 7F show other embodiments of light delivering elements 32E′, 32F′ having a shape or profile geometry that restricts rotation once inside the inner lumen of the housing 22E′, 22F′. As illustrated, the inner lumen of housing 22E′, 22F′ can form a keyhole-like shape, while the outer shape of the housing 22E′, 22F′ can be substantially cylindrical.

The housing can be made from a variety of materials including polymeric, electrically nonconductive material, like polyethylene (PET), polytetrafluoroethylene (PTFE), FEP, PFA, urethane, polyurethane, or PVC, which can withstand tissue coagulation temperatures without melting and provides a high degree of laser light transmission. Preferably, the housing is made of Teflon® tubes and/or coatings. The use of Teflon® improves the procedures by avoiding the problem of fusion or contact-adhesion between the ablation element and the cardiac tissue during usage. While the use of Teflon® avoids the problem of fusion or contact-adhesion, the hand held cardiac ablation instrument does not require direct contact with the tissue to effect a therapeutic or prophylactic treatment. Preferably, the housing incorporates opaque or semi-opaque materials such as ePTFE, and/or includes optically transparent windows that provide for light transmission.

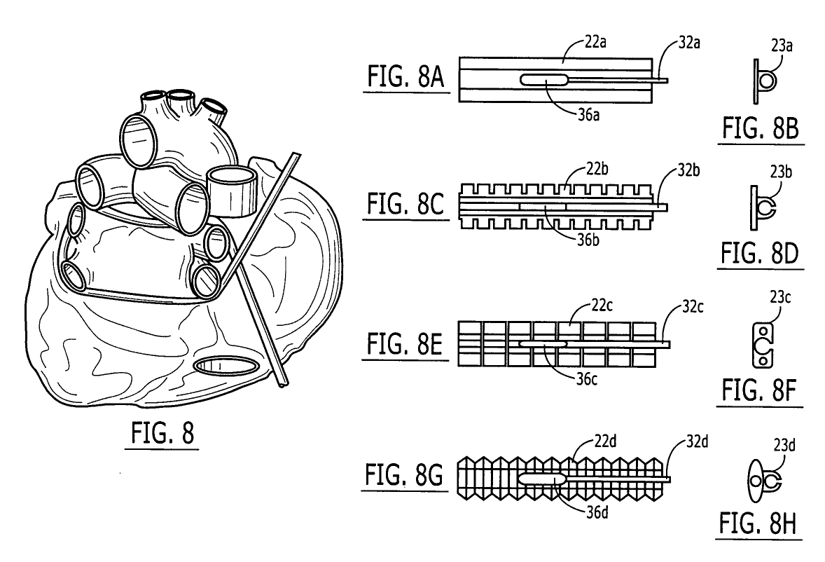

The housing is designed with longitudinal flexibility to ensure adequate conformance to various tissue topographies. For example, as shown in FIG. 8, a flexible housing enables the ablation instrument to adequately contact the cardiac tissue around the pulmonary veins. In addition to longitudinal flexibility, the housing can be configured to have torsional stiffness characteristics as well to resist twisting. Resistance to twisting ensures that the ablative energy is directed toward the desired target tissue to maximize the effectiveness of the ablation and to minimize collateral damage. Because much of the housing is not visible to the surgeon during use because the left atria is located on the posterior surface of the heart, it is therefore important that the housing ensure both adequate contact and rotational alignment with the target tissue to provide effective ablation.

To provide the housing with longitudinal flexibility as well as anti-twist or torsionally stiff properties, materials such as PTFE, PFA, FEP, urethane, or PVC can be used. Other similar materials can also be used which have flexural modulus properties, profile, reinforcement, or filler materials that resist twisting along the longitudinal axis. By combining various structural elements and material properties, the housing can resist twist and remain straight in two planes. In addition, by providing an element that is shaped in three dimensions inside the housing, it is possible to provide adequate positioning and flexure within difficult anatomical locations. For instance, the shaped element could include stainless steel, Nitinol (NiTi) or polymer round or flat wire pre-shaped to a desired shape or geometry. This shaped element could also include a malleable stainless steel or polymer structure that is manipulated by the surgeon to provide the desired positioning, as previously described in the embodiment of FIGS. 6 and 6A. In an alternative embodiment, the housing can be provided with a series of inflatable chambers (not shown) to effect the desired shape and/or remove twist from the structure.

In still a further embodiment, the housing can include a channel or lumen that, once positioned proximate to the target tissue, can be filled with a setting agent such as epoxy, UV cured adhesive, thermosetting polymer, or other material that can be inserted in liquid or gel form into the channel or lumen that, when cured, provides a rigid structure to the housing. This rigid structure then provides proper shape and position to the housing during the procedure. Alternatively, a thermoplastic metal, polymer or liquid that hardens and softens at specific temperatures can be applied to provide for a rigid structure. Following the ablation process, the filling material can be dissolved, melted, broken down, or otherwise removed to return the housing to its original flexible form for removal.

Further, the housing of the present invention can include a profile that provides for longitudinal flexibility and proper orientation with respect to the target tissue to be ablated. As illustrated, FIGS. 8A-8H show a variety of profile designs for the housing 22a, 22b, 22c, 22d, as well as profile designs for the reflector 23a, 23b, 23c, 23d in accordance with the invention. FIGS. 8A, 8C, 8E, and 8G show that the housing 22a, 22b, 22c, 22d can be formed of an optically clear material and can be formed as an integral unit, or as discrete units linked together. The housing 22a, 22b, 22c, 22d can also include grooves to facilitate flexion. In addition, the housing 22a, 22b, 22c, 22d can be formed as a bellows to allow bending. FIGS. 8B, 8D, 8F, and 8H show that the reflector 23a, 23b, 23c, 23d can have a three-dimensional profile that allows the placement of the light delivering element 32a, 32b, 32c, 32d inside the housing 22a, 22b, 22c, 22d in only one direction. For example, the profiles can include “D” shapes, half moons, open “C” channels, or other similar configurations that would align with the inner lumen of the housing 22a, 22b, 22c, 22d in a specific orientation, as previously described for FIGS. 7A-7F.

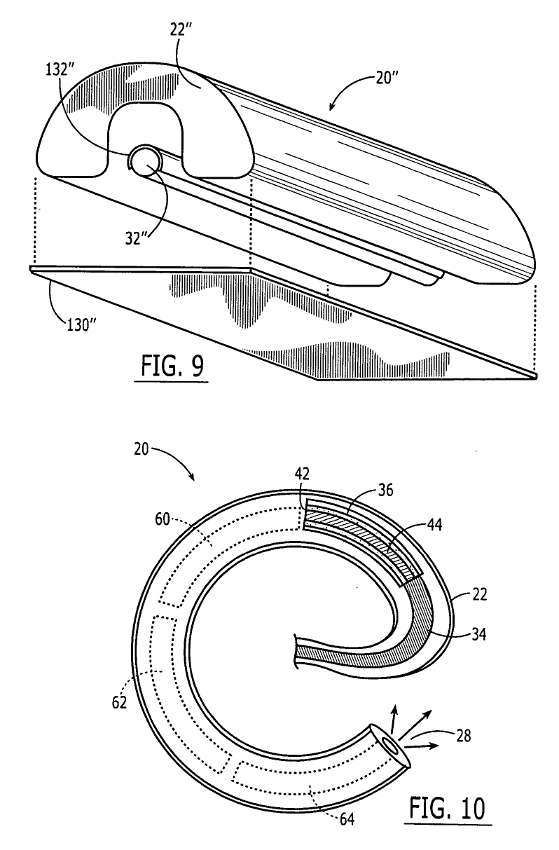

In another embodiment of the present invention, rather than rely on the profile geometry for alignment of the light delivering element with the housing, reflective elements can be implemented which would eliminate the need for such specific geometries. As shown in FIG. 9, a housing 22″ is shown having an open “C” shape to define an inner lumen within which a light delivering element 32″ is slidably contained. A “C” shaped reflector 132″ is placed over the light delivering element 32″ to isolate the emission of ablative energy to the uncovered portions. This ablative energy can be transmitted through a light transmissive sheet 130″ placed over the housing 22″ and onto the target tissue. The reflector can be formed from metallic foils, polymers with highly reflective surfaces, vapor or chemical deposited surfaces, or other materials having a reflective or mirror-like surface.

Although illustrated in the context of light delivering surgical instruments, the malleable structures disclosed herein are equally adaptable for use with other sources of ablative energy, such as such as RF heating, cryogenic cooling, ultrasound, microwave, ablative fluid injection and the like. RF Heating devices, for example, are described in U.S. Pat. No. 5,690,611 issued to Swartz et al. and herein incorporated by reference. Cryogenic devices are similarly described, for example, in U.S. Pat. No. 6,161,543 issued to Cox et al. and herein incorporated by reference.

Epicardial ablation is typically performed during a by-pass procedure, which involves opening the patient's chest cavity to access the heart. The heart can be arrested and placed on a by-pass machine, or the procedure can be performed on a beating heart. The hand held ablation instrument 10 is placed around one or more pulmonary veins, and is preferably placed around all four pulmonary veins. The connecting element 30 can then be attached to the distal end 16 of the handle 12 or the proximal, trailing end 24 of the outer housing 22 to close the open loop. The handle 12 can optionally be pulled to tighten the ablation element 20 around the pulmonary veins. The energy delivering element 32 is then moved to a first position, as shown in FIG. 10, and the energy source 50 is activated. The first lesion is preferably about 4 cm in length, as determined by the length of the tip 36. Since the distance around the pulmonary veins is about 10 cm, the energy delivering element 32 is moved forward about 4 cm to a second position 60, shown in phantom in FIG. 10, and the tissue is ablated to create a second lesion. The procedure is repeated two more times, positioning the energy delivering element 32 in a third position 62 and a fourth position 64. The four lesions together can form a lesion 48 around the pulmonary veins, for example. Advancement in such a manner includes a certain amount of overlap between the initial position and the advanced position. Typically, for a 5 cm long ablation element 20, the instrument 10 might be advanced 4 cm at a time to thereby create a series of local 1 cm lengths that experience double therapies, ensuring a continuous lesion.

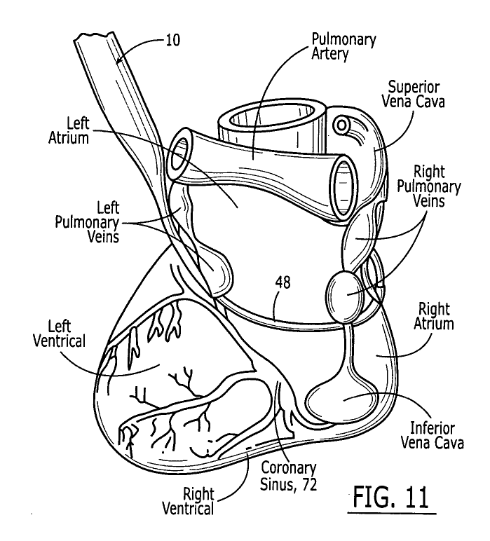

In another aspect of the invention, the instruments of the present invention are particularly useful in forming lesions around the pulmonary veins by directing radiant energy towards the epicardial surface of the heart and the loop configuration of distal end portion of the instruments facilitates such use. It has been known for some time that pulmonary veins can be the source of errant electrical signals and various clinicians have proposed forming conduction blocks by encircling one or more of the pulmonary veins with lesions. As shown in FIG. 11, the instrument 10 of the present invention is well suited for such ablation procedures. Because the pulmonary veins are located at the anterior of the heart muscle, they are difficult to access, even during open chest surgery. An open loop distal end is thus provided to encircle the pulmonary veins. The open loop can then be closed (or cinched tight) by a clasp, as shown. (The clasp can also take the form of suture and the distal end of the instrument can include one or more holes to receive such sutures as shown in FIG. 2.) The longitudinal reflector structures described above also facilitate such epicardial procedures by ensuring that the light from the light emitting element is directed towards the heart and not towards the lungs or other adjacent structures.

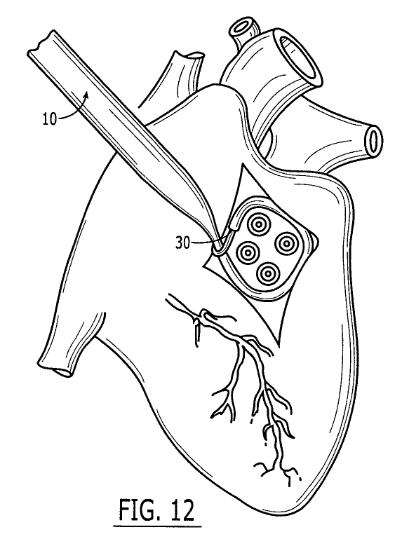

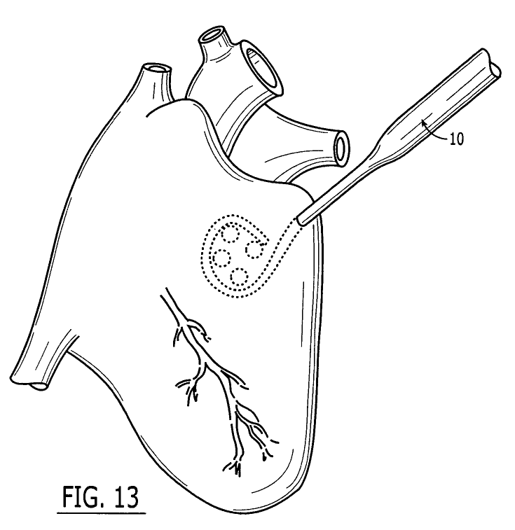

Endocardial applications, on the other hand, are typically performed during a valve replacement procedure which involves opening the chest to expose the heart muscle. The valve is first removed, and then the hand held cardiac ablation instrument 10 according to the present invention is positioned inside the heart as shown in FIG. 12. In another approach the instrument 10 can be inserted through an access port as shown in FIG. 13. The ablation element 20 can be shaped to form the desired lesion, and then positioned at the atrial wall around the ostia of one or more of the pulmonary veins. Once shaped and positioned, the laser energy source 50 is activated to ablate a first portion of tissue. The light delivering element 32 can then be slidably moved, as described above with respect to the epicardial application, or alternatively, the entire device can be rotated to a second position to form a second lesion.

In another aspect of the invention, the ablation element 20 can be configured to have a sufficient length to create the full encircling path without advancing the light delivering element 32 through the outer housing 22. For instance, the ablation instrument 10 can include a long (20 cm) active length that can emit at the same energy level (W/length) as that delivered by the shorter (5 cm) instrument, or can emit at a lower level. To provide effective ablative therapy, an adequate quantity of Joules per volume of tissue should be delivered. The rate of delivery, however, can be adjusted depending upon the capabilities of the materials and components of the ablation instrument 10. Thus, the length of the ablative element 20 and consequently, the time required to complete the ablative therapy, can be varied without affecting the integrity of the overall ablation process.

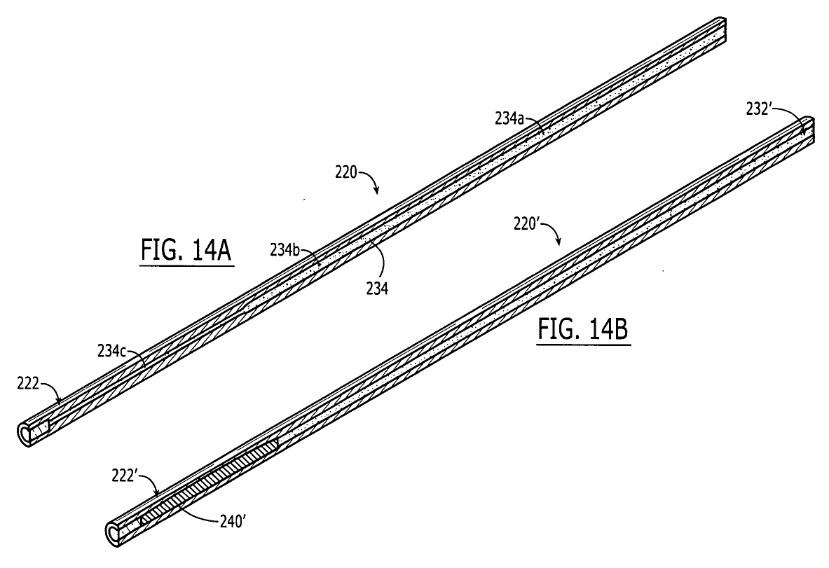

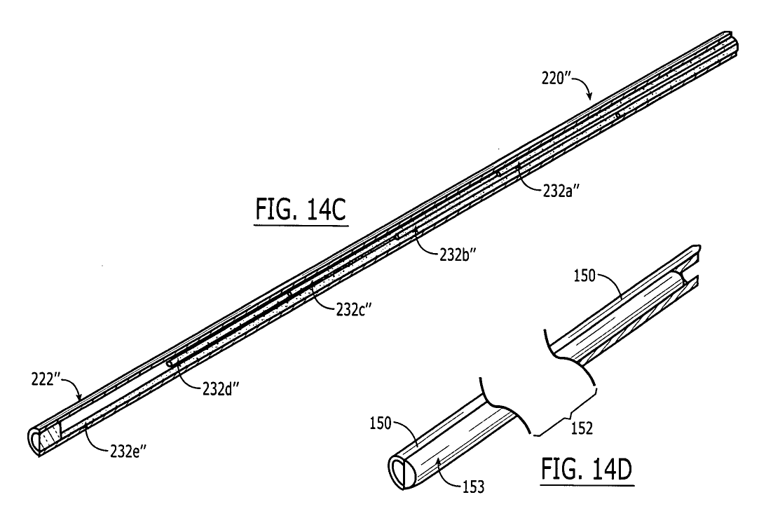

Accordingly, it is possible to provide a light delivering element 32 that can emit varying amounts of ablative energy along its length. FIGS. 14A-14D illustrate such ablation elements 220, 220′, each of which are configured with a length sufficient to provide a continuous encircling lesion without the need for repeated advancing of the light delivering element 232, 232′ to create successive therapies along the ablative path. For example, in one particular embodiment, the optical fiber 234 can have a varying diameter along its length. As shown in FIG. 14A, a first section 234a has a greater diameter than an adjacent second section 234b, which has a greater diameter than an adjacent third section 234c of the optical fiber 234. In the embodiment shown, the light delivering element 232 comprises a plurality of segments, each segment having a different diameter than an adjacent segment to collectively form an elongate energy emitting element having variable diameters along its length. The light delivering element 232 can also be provided with a tapered profile along its length, in order to vary the amount of ablative energy emitted.

In another embodiment of the ablation element 220′ shown in FIG. 14B, an inflatable elongate balloon 240′ can reside within the housing 222′ along with the light delivering element 232′. An inflation controller in communication with the balloon 240′ and an inflation source, e.g., an air, gas or fluid pump, can be provided to enable the selective inflation of the balloon 240′. Upon inflation, the balloon 240′ can be configured to urge against the light delivering element 232′, causing the angular orientation of the optical fiber 234′ to adjust with respect to the longitudinal axis of the housing 222′. Thus, by selectively inflating and deflating the balloon 240′, the surgeon can change the angle of the light delivering element 232′ and consequently the energy emitting pathway along its length.

In yet another embodiment shown in FIG. 14C, the ablation element 220″ can be provided with a plurality of light delivering elements 232″ of varying lengths to deliver a fraction of the total ablation energy to different areas along the length of the ablation element 220″. FIG. 14C illustrates a housing 222″ containing six light delivering element 232a″-232e″, e.g., optical fibers; however, it is understood that any number of fibers 232a″-232e″ can be utilized as needed. Because the total ablation energy being delivered is fractionated, each of the fibers 232a″-232e″ has a smaller diameter than would be required for a single optical fiber 232a″-232e″ delivering the same total amount of energy. Therefore, the fibers 232a″-232e″ are more flexible, resulting in an overall more flexible ablation element 220″.

Another way to change the level of ablative energy being delivered by the ablation element is to selectively block or cover areas along the length of the light delivering element. For example, as illustrated in FIG. 14D, a reflector 150 having a window 152 or discontinuous outer surface formed from a metallic or reflective material, such as gold, can be applied over a light delivering element 153. The reflector 150 can be configured to seat around the light delivering element 153, and a window can be provided to allow the emission of ablative energy from the light delivering element 153. The window 152 can be adjustably moved along the length of the light delivering element 153 to effect a change in the level of ablative energy being delivered along the length of the light delivering element 153. Alternatively, it is also possible to provide a reflector 150 having an adjustably sized window 132 whereby the surgeon can control the amount of exposure to adjust the level of emitted ablative energy of the light delivering element 153.

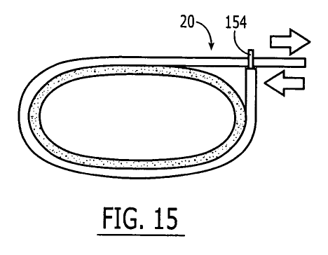

Whether the ablation instrument 10 requires advancement or is completely encircling, there is a potential need to provide overlap of the ablation at either end of the outer housing 22. A clamp or clip mechanism 154, as shown in FIG. 15, can be provided to fix the outer housing 22 at both ends in order to ensure that both ends of the therapeutic lesion overlap for a continuous encirclement. Of course, other configurations are also possible to connect or enable overlap of the two ends of the outer housing 22, as previously described in connection with FIGS. 2 and 12. It is also possible to increase the time for ablation at the overlap to better ensure a completely encircling lesion has been formed.

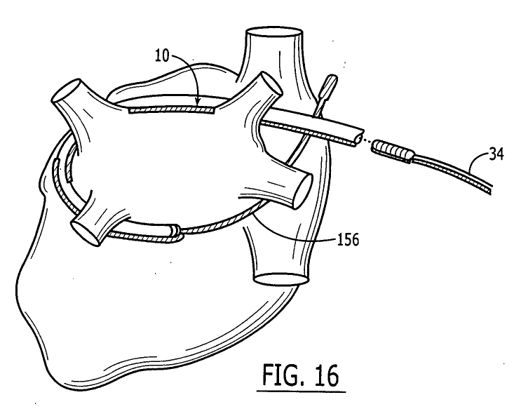

As discussed above, correct positioning of the housing 22 with respect to the patient's anatomy is critical to the efficacy of the lesion created. Specifically, the position of the housing 22 with respect to the left atrial appendage (LAA) is important to ensure that the lesion correctly isolates the pulmonary veins. The correct position of the housing 22 in such a procedure should be posterior to the LAA or between the LAA and the pulmonary vein. Through specific surgical approaches such as thoracotomy, thorascopy, sternotomy, sub xyphoid, or other undetermined surgical or scoped approaches, delivery and positioning of the housing 22 may require additional verification of position with respect to the LAA. Accordingly, the ablation instruments 10 of the present invention can incorporate radiopaque or echogenic ultrasound visible coatings or components. In addition, the application of radiopaque markers/dyes to the blood volume with techniques such as transesophageal echocardiograms (TEE) or fluoroscopy can provide further confirmation of the position of the housing 22. In more invasive procedures, a thorascope can be used to obtain visual confirmation from the left chest. Other less invasive methods include the use of impedance measurements between electrodes and the housing, or shaped introducing guides 156 that provide for preferential positioning of the housing, as shown in FIG. 16.

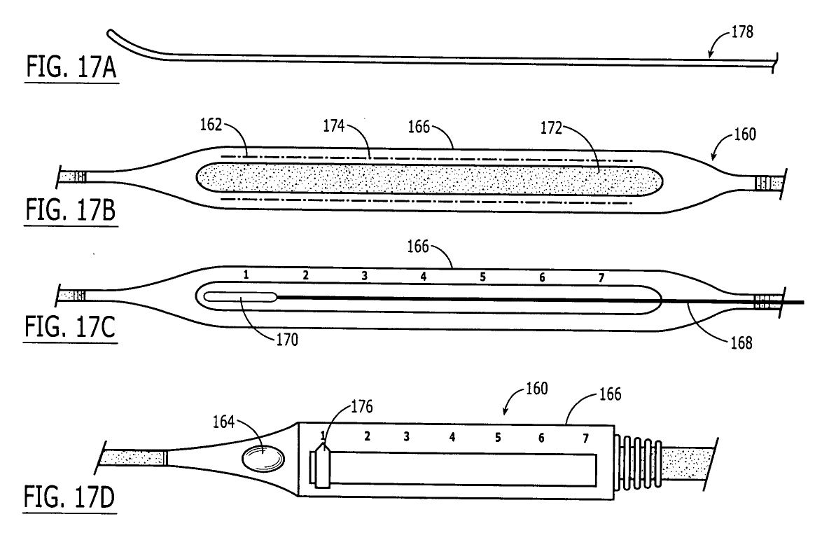

FIGS. 17B-17D illustrate another embodiment of an ablation instrument 160 of the present invention. As shown in FIGS. 17B and 17D, the ablation instrument 160 includes conduction block sensors 162 and a conduction block indicator 164 on the housing 166 for determining the effectiveness of the lesion created. These sensors can be integrated into or attached to the housing 166. In the particular embodiment shown, the ablation instrument 170 includes a single, slidable light delivering element 168 extending into a diffuser tip 170. The housing 166 can include a window 172 to allow ablative energy to be emitted, and a plurality of irrigation ports 174 to introduce irrigation fluid into the housing 166 to cool the instrument 160. Similar to the previous ablation instruments 10 described for FIGS. 1 and 2, the light delivering element 168 can be moved along the length of the housing 166 by a translatory mechanism (as previously shown). As illustrated in FIGS. 17C and 17D, indicia 176 along the window 172 provides a visual cue for the surgeon to determine how far the light delivering element 168 has moved. The ablation instrument 160 can be used with a shaped, flexible guidewire 178 as shown in FIG. 17A.

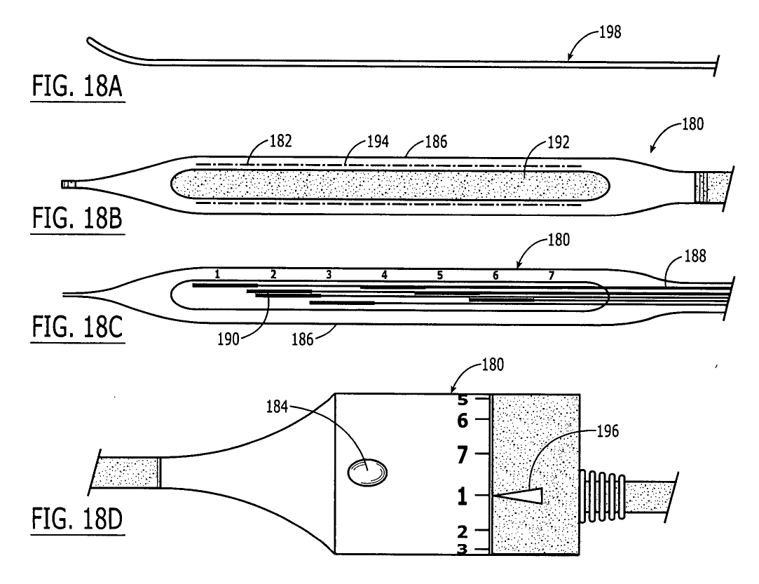

FIGS. 18A-18D show a similar ablation instrument 180 but with a plurality of light delivering elements 188 of varying lengths. Similar to FIGS. 17B and 17D, the ablation instrument 180 includes conduction block sensors 182 and a conduction block indicator 184 on the housing 186 for determining the effectiveness of the lesion created, as shown in FIGS. 18B and 18D. Each of the slidable light delivering elements 188 extends into a diffuser tip 190. The housing 186 can include a window 192 to allow ablative energy to be emitted, and a plurality of irrigation ports 194 to introduce irrigation fluid into the housing 166 to cool the instrument 160. The light delivering elements 188 can be selectively chosen by a rotatable selection mechanism 196 which includes indicia which includes markings to indicate which of the elements 188 has been chosen. The ablation instrument 180 can be used with a shaped, flexible guidewire 198 as shown in FIG. 18A.

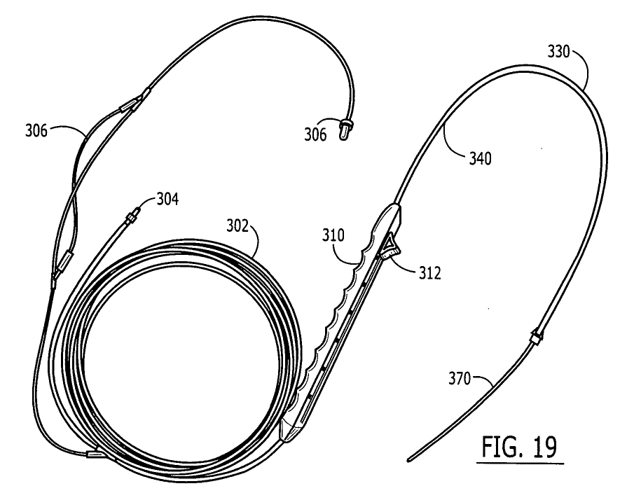

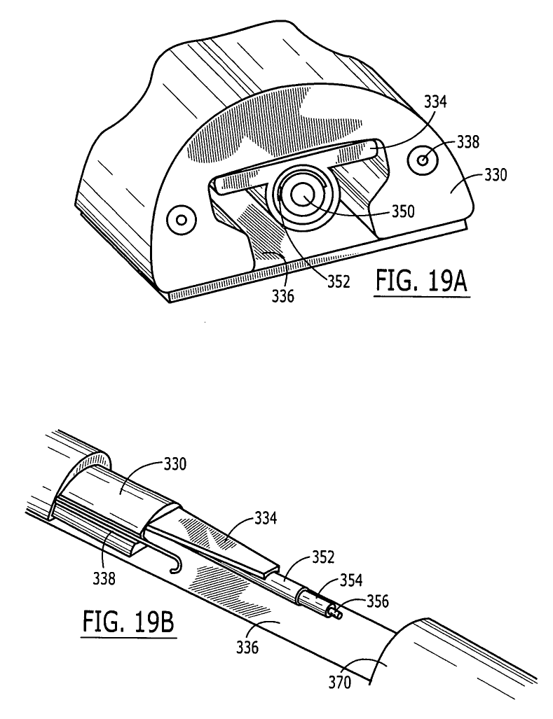

In still yet another embodiment, the present invention provides an ablation instrument 300 that can incorporate many of the advantages and features of the previous embodiments described above. As illustrated in FIG. 19, the ablation instrument 300 can include a handle portion 310 having a flexible sheath 330 coupled thereto. The flexible sheath 330 can connect to the handle portion 310 by way of an extension 340. Within the sheath 330 is an ablation element 350 that can be connected to the handle portion 310 and that is moveable along an ablative path or lumen 332 inside the sheath 330 via movement of the indexing button 312 located on the handle portion 310. The sheath 330 can extend into an atraumatic guide 370 at the tip, or opposite end, of the instrument 300.

As shown, a cable 302 extends from the ablation element 350 and handle portion 310 to an attachment device such as a cable connector 304 which is adapted to be received by an energy source such as a laser source. Also extending from the cable 302 is an irrigation line 306 which allows the instrument 300 to receive irrigation fluid. The irrigation line 306 can include an attachment device, such as a male luer lock 306, for attachment to an irrigation fluid source.

The sheath 330 of the ablation element 350 can have a variety of configurations, and the sheath 330 may be preshaped or flaccid. In an exemplary embodiment, the sheath 330 is adapted to function as a guide device to direct the ablation element 350 along the treatment path, and more preferably it can be adapted to cooperate with the ablation element to position the ablation element in a proper orientation to facilitate ablation of the targeted tissue during the advancement. Thus, once the ablation sheath 330 is stationed relative to the targeted contact surface, the ablation element 350 can be easily advanced along the ablation path to generate the desired tissue treatment. The sheath 330 can also serve as an energy shield to protect tissues not targeted for treatment.

FIGS. 19A and 19B illustrate one exemplary embodiment of the sheath 330. As shown, the sheath 330 has an inner lumen 332 extending therethrough for slidably receiving the ablation element 350, and an optically transmissive window 336 formed along at least a portion thereof. The ablation element 350 includes a fiber having a diffuser 354 disposed therearound, and a reflective element 352 disposed on a portion thereof for reflecting emitted energy toward a target ablation site. The inner lumen 332 of the sheath 330 has a shaped profile or special geometry that is adapted to receive an ablation element 350 having a shaped profile that substantially complements the shaped profile of the lumen. While the shaped profile can vary, in the illustrated exemplary embodiment, the sheath 330 is substantially D-shaped, and the ablation element 350 includes a T-bar shaped spine element 334 formed thereon and adapted to be received within the inner lumen 332 of the sheath 330. The T-bar shape of the spine element 334 will prevent rotation of the ablation element 350 within the lumen 330. Thus, since the ablative instrument 300 is designed to directionally emit the ablative energy from a select area of the instrument called the energy delivery portion, the spine 334 allows the ablation element 350 and the sheath 330 to be aligned to assure that the correct directionality of emitted ablative energy toward the tissue region is emitted.

The sheath 330 may be made of a variety of materials, but one exemplary material is ePTFE. The porosity, density, pore size and other physical characteristics of the material should be selected so as to improve the performance of the sheath. These characteristics should be carefully chosen to give the best combination of longitudinal flexibility, tissue conformability, torsional resistance, lubricity, atrauma and shielding. Preferably, the sheath 330 is made from a polymeric material, like polyethylene, PTFE, PTFA, FEP or polyurethane, which can withstand tissue coagulation temperatures without melting and to provide a high degree of laser light transmission. Alternative designs of the sheath may incorporate opaque or semi-opaque materials such as ePTFE that incorporate optically transparent “windows,” such as window 336, providing for light transmission. The spine element 334 is preferably formed by extrusion in PEBAX polymer.

The sheath is preferably designed with longitudinal flexibility to insure adequate contact with cardiac tissue, but it can also have torsional stiffness characteristics to resist twisting. Resistance to twisting insures that the ablative energy is directed only toward the desired tissues so to maximize ablative effectiveness and to minimize collateral damage. Alternative designs may rely upon uniquely shaped profiles and torsional flexibility to allow conformance to the variant tissue topographies. Much of the sheath is not visible to the surgeon during use because the left atrium is located on the posterior surface of the heart and there is additionally other anatomy such as the pericardium and great vessels in close proximity. Without preferred visualization of the sheath it is therefore important that the sheath ensure both adequate contact and rotational alignment with the target tissue.

Another feature of the sheath 330 is its anti-twisting properties, which relate to the ability to correctly orientate a device that is required to be rotationally directed towards a target while traveling through a flexible linear path with a window capable of being translucent to the specific energy. The mechanism of the invention is to create loosely interlocking geometries that interact to prevent rotational displacement. These components are then utilized to fix a therapeutic device within one or both of these components such that directional orientation is assured. As shown in FIG. 19A, the T-shaped spine element 334 interacts within the larger “T” shape channel (externally “D” shaped) of the lumen 332 to properly align a reflector 352 of the therapeutic device towards the clear therapy window 336. The sheath 330 can also include stabilizers 338, such as Nitinol (NiTi) flat wire, polymer ribbon, or protruding devices adhered or incorporated into the profile thereof to interact with the guide sheath 330 and limit the capability of the ablation device 350 to rotate within the sheath 330. The stabilizers 338 can also be adapted to provide a shielding effect and/or a reflective effect to direct energy toward the window 336. Thus the shape of the stabilizers 338 can vary depending on the intended purpose.

Preferred embodiments of the disclosed invention including anti-twist or torsionally stiff properties include profiles of PTFE, PFA, FEP, Urethane, PVC or other similar materials that by properties such as flexural modulus, profile, reinforcement, or filler materials result in a sheath that resists twist along the longitudinal axis. By combining various structural elements and material properties it is further possible to provide for a device that resist twist and remains straight in two planes or is preferentially shaped in three dimensions. By providing a three dimensionally shaped element within the sheath it is possible to provide adequate positioning within even the most variant anatomy.

Yet a further embodiment of the current disclosure would include a channel or lumen within the sheath that once in position would be filled with a material such as epoxy, UV cured adhesive, thermosetting polymer or other material that can be inserted in liquid or gel form into such lumen or channel and when cured provides a rigid structure to the sheath. This rigid structure then provides proper shape and position to the sheath during the procedure. Alternately the material could be a thermoplastic metal, polymer, or liquid that hardens and softens at appropriate temperature and provides for similar structure. Following the therapy process the filling material would be dissolved, melted, broken, or otherwise affected to destroy the previous rigid structure and return the sheath to a flexible form for removal.

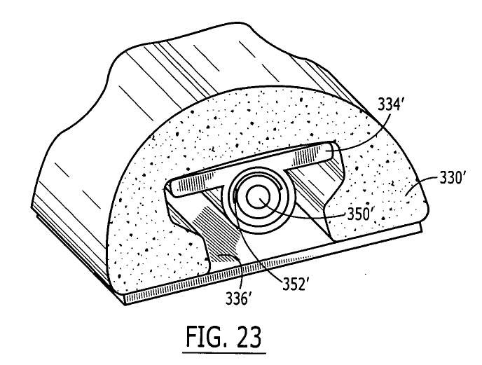

In another exemplary embodiment, the sheath 330 can be extruded with a shielding material, such as a dye or particulate to focus the energy toward the window 336. For example, by utilizing metallic particulates as a loading agent in the material it would be possible to adequately shield an RF or ultrasound antennae to create a directional emission of energy. FIG. 23 illustrates a sheath 330′ having particulate embedded therein to create a shielding effect. While a reflector 352′ is shown disposed on the spine 334′, the particulate may be effective alone to shield the energy, and thus a reflector 352′ may not be necessary.

Anti-twist designs may further include preferable profiles of the sheath that rely upon the shape of the profile rather than torsional rigidity to provide correct alignment with the target tissue. Such preferred profiles would include “D” shapes, half moons, open “C” channels, triangular channels, or other similar and varied designs that interact to align the light delivering element with the tissue. The preferred embodiment of the current disclosure is a “D” shape whereby the flat segment of the “D” provides such accurate alignment with the tissue when coupled with a sheath material that is torsionally flaccid. The crown of the “D” further provides for visual or tactile verification of alignment.

The previously described embodiments providing for anti-twist or alignment of the sheath could incorporate reflective elements that would eliminate need for the above described “special geometry” that operates to align the light emission device. By providing reflective elements on the guide sheath it would therefore be possible to eliminate the directional orientation device on the ablative device. The reflective element(s) could also be provided on the spine 334′, as shown in FIG. 23, to allow the energy emitting device, e.g., the fiber 350′, to rotate freely within the spine 334′. With such a configuration, the spine 334′ can form a catheter or guide tube for the energy emitting device, and the spine 334′ interacts with the sheath 330 to position the reflective element(s) in the proper orientation. As shown in FIG. 23, a reflective element 352′ is disposed within the lumen of the spine 334′ to direct energy toward the window 336′. While not shown, the spine 334′ can have a curved configuration or other shapes that allow the reflective element 352′ to direct energy toward the window 336′. The reflective element 352′ could also be disposed within the spine 334′ itself, rather than in the inner lumen. Diffuser 354 can also include a mirror 356, as shown in FIG. 19B.

Such reflective elements could include but are not limited to metallic foils, polymers with highly reflective surfaces, vapor or chemically deposited surfaces or other technologies that result in a reflective or mirror like surface. The advantage of this system over the prior art is that the energy emissive element is not required to be shaped to match the channel. Rather, the positioning component can be shaped appropriately and the energy emission element can then be fixed to this component, or it can be slide and/or rotate freely within this component. By attaching the reflector 352 to the positioning component, e.g., the spine 334 or the sheath 330, rotation of the energy transmitter is irrelevant to the energy emission direction. This is beneficial in that the emitter does not require a shaped output, rather the alignment feature directs this output.

The second advantage of this invention is the novel use of FEP and ePTFE to create an insulating and transmissive guide channel. This is advantageous over prior art in that the addition of FEP creates an optically clear window 336. In an exemplary embodiment, the sheath 330 includes a hemi-cylindrical portion formed from ePTFE, and a planar bottom surface formed from FEP that are bonded together using heat and pressure to form the D-shaped sheath 330. Further, it is notable that this same technology could be utilized for endoscopic evaluation of anatomical structures whereby a endoscopic evaluation device may be passed down the length of the channel and visually inspect the tissues in contact with the guide channel. This may be of great advantage when tissues in opaque or visually impeding fluids typically surround the structure to be treated. The ability to particular or pigment load (using multicolored extrusion lines) the alignment spine 334 in order to create either electromagnetic shielding and/or optical shielding for controlling the emissive aperture is also an additional feature of the present invention. Also, the present invention provides the ability to create an optical lens on the spine 334 to create a focused energy emission. Specifically, by bulking up or shaping the segments of the tubing, it would be possible to create a focusing or diverging lens to create the appropriate emission.

Thirdly, the creation of a T-shaped shrink tube provides the ability to appropriately pass coolant throughout the length of the channel as well as providing proper orientation. In addition, the sheath 330 bears graphical markings and numberings to aid the surgeon in orienting and positioning the device on cardiac tissue. The markings and their blue color were specifically designed to enhance visibility and recognition under operating room lighting conditions. Further, a transmurality sensor or other lesion effectiveness/assessment sensor may also be integrated into or attached to the sheath.

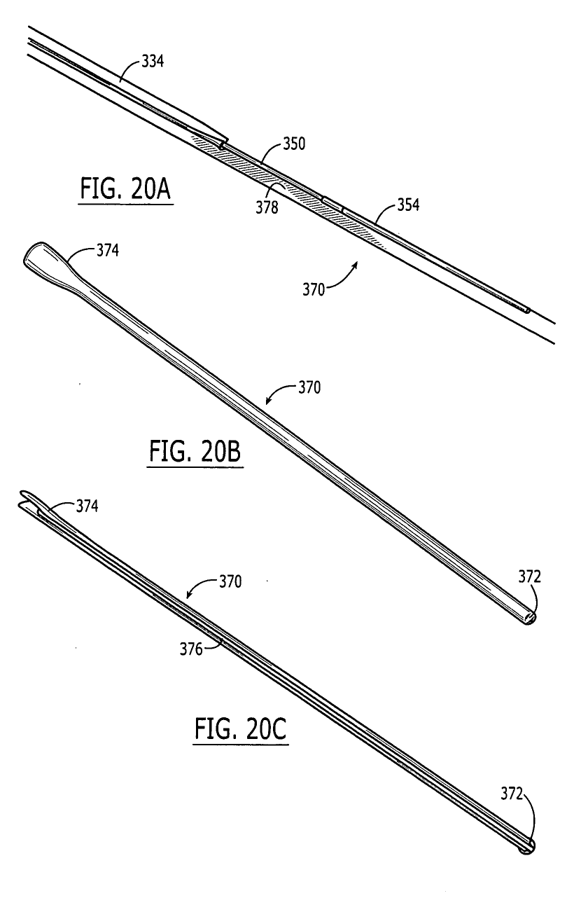

Turning now to another component of the ablation instrument 300, FIGS. 20A-20C illustrate the flexible tip or guide 370. FIG. 20A illustrates an exploded view of the atraumatic tip 370, which also includes a window 378 for energy emission. As shown, the spine 334 enables the ablation element 350 and diffuser 354 to be slidably extended through its lumen 376. As shown in FIGS. 20B and 20C, the guide 370 includes a blunt, atraumatic tip 372 and a flared extension 374 at an opposite end for creating an atraumatic connection with the sheath 330. Extending longitudinally within the guide 370 is a lumen 376 for slidably passing the spine 334 and ablation element 350.

The guide component design is optimized to provide minimal trauma and resistance during surgical placement while providing maximum visibility under OR lighting and maximal grip by forceps and other surgical instruments. Its dimensions, geometry and material are specifically chosen for this purpose. Its design includes both an external flat for easy visual and tactile orientation during use, and an internal channel designed to provide an optimal feel to the surgeon. The guide is an injection molded component, made of a TPE. It includes an integral connector which allows it to be bonded to the distal end of the sheath with a UV adhesive. Its surgical “feel” is enhanced by its closed end, hollow cylindrical design. This internal feature is created through use of a wire placed in the mold prior to injection and removed after part molding is complete. The tip of the cylinder is closed by an RF heat forming process. Although the external cross section of the guide is essentially round, it does include a flat on its bottom side. This flat serves to improve the feel that the surgeon perceives when grasping the guide with surgical instruments. The exterior surface of the guide bears a no slip matt finish, rather than a polished finish, to improve the surgeons ability to easily grip the part with his instruments.

The integral connector is designed to also function as an atraumatic means of transition from the small cross section guide to the larger cross section sheath. This feature is important since the device also dilates and separates the sometimes fragile cardiac tissues during surgical placement.

The device's extension 340 is specifically designed as a flexible, rather than rigid component. This approach makes the instrument 300 both more ergonomic for the surgeon and less obtrusive in the crowded surgical field. It is formed of an extrudable polymer and contains helically wound stainless steel wire to prevent kinking when flexed. This component serves two functions. It provides room for the 7 cm movement of the therapeutic fiber 350 as it is indexed forward and backward. It also provides physical separation between the light delivering sheath 330 and the handle 310. This separation makes the instrument 300 more easily and conveniently used in the always crowded sterile field. It allows a more ergonomic positioning of the handle relative to the surgical access site, including angular orientations.

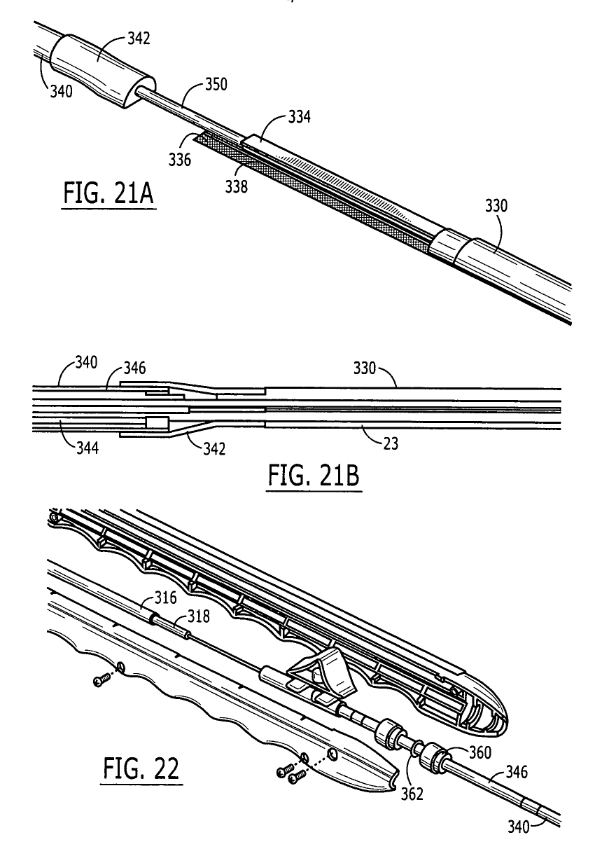

In one preferable embodiment, the extension 340 is bonded to the sheath 330 with UV cured adhesive using a molded thermoplastic connector. The extension 340 can be attached to the sheath with a sheath connector 342, as shown in FIGS. 21A and 21B.

The instrument 300 includes a handle 310 attached to the sheath 330. An inner lumen can extend through the handle to receive the light delivering element 350. The passing of the light delivering element is performed by incrementally advancing the ablative element 350 along a plurality of positions of the ablation path to produce a substantially continuous lesion.

Ablation with a continuous encircling lesion in the current disclosure is intended to occur by advancing a short, perhaps 1-5 cm long, ablation device that is repetitively positioned, activated, and advanced to create successive therapies along the path of the guide sheath. Advancement includes a certain amount of overlap between the initial position and the advanced position. For example a 5 cm long device might be advanced 4 cm at a time thereby creating a series of local 1 cm lengths that experience double therapies. In this manner a continuous lesion set can be insured.

The handle 310 is designed to allow comfortable, one handed indexing. The indexing button 312 and mechanism provide very positive tactile and audible feedback to the user when each index location is reached. Among other benefits, this design allows the surgeon to effectively index the device without looking at the handle. The surgeon is able to track the location of the ablative diffuser by the feel and sound of the handle's feedback mechanism. The surgeon is also able to visually locate and track the position of the ablative element within the sheath by observing the red glow of device's red aiming beam, which is visible through the shield side of the sheath 330.

The handle 310 has an overall triangular cross section designed to ergonomically fit the surgeons hand. It also includes multiple finger grips which aid single handed actuation of the indexing button 312. The audible and tactile responses are created through use of a spring loaded ball detent assembly 314 contained in the indexing button 312 and corresponding slots formed in the handle at each indexing position.

The handle 310 is sequentially marked by numbers 1-7, one number at each index position. These numbers correspond to the ablating element indexing positions also marked on the sheath. The handle 310 also includes a dynamic o-ring seal which functions to contain the irrigation fluid inside the device while allowing easy indexing.

Alternative embodiments of the device may include long (20 cm+) active lengths that are placed and left in position to create the full encircling path without advancing the device through the guide sheath. This may be enacted at the same dose level (perhaps W unit length) as that delivered by the shorter (4 cm) device or may alternatively be a significantly lower dose. It is believed that a quantity of Joules per volume of tissue must be delivered in order to provide an effective therapy. Therefore the rate of delivery of this energy can be accelerated or slowed depending upon the capabilities of the materials and components therefore allowing the use of various configurations to provide different active lengths. The variable that would be changed to control the amount of energy delivered would then be therapy time.

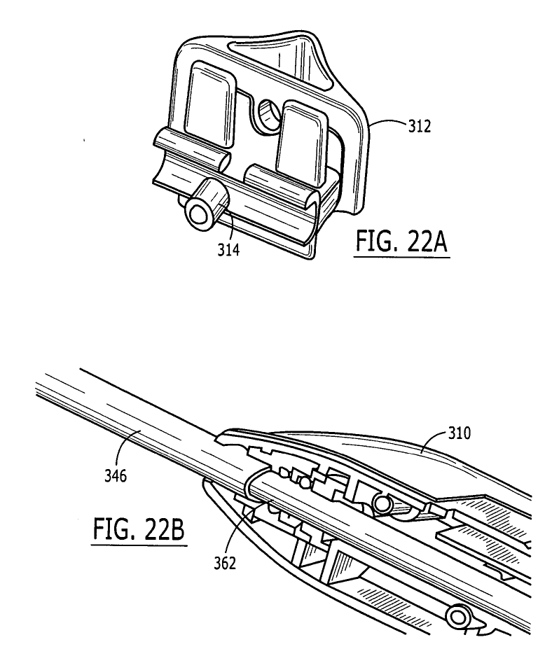

FIG. 22 illustrates an exploded view of the handle portion 310 of the ablation instrument 300. As shown, the extension 340 is attached to an indexing button 312 by means of an inner extension 346. The inner extension 346 can be configured within an o-ring housing 360 between which there is an o-ring 362 for seating within the handle portion 310. An outer fiber cover 316 and inner fiber cover 318 envelope the ablation element or fiber 350, which extends into a flow channel 344 connected to the inner extension 346. Seated on the exterior of the flow channel 344 is the indexing button 312, which includes a ball detent assembly 314 as shown in FIG. 22A. By exerting a downward pressure against the indexing button 312, the surgeon is able to effect linear movement of the flow channel 344 which then moves the ablation element 350. FIG. 22B illustrates an alternative embodiment of the handle portion 310 in which the flow channel 344 is attached to a single o-ring 362 to form a seal near the inner extension 346.

As shown in FIG. 19, the ablation instrument 300 of the present invention also utilizes an irrigating fluid. An irrigating fluid is disposed between the light delivery element 350 and the sheath 330. This fluid is a physiologically compatible fluid, such as saline, and is used to cool the light emitting element and for tissue irrigation via one or more exit ports in the sheath 330.

Irrigation serves to increase the efficiency and effectiveness of the device by acting as an optical couple between the diffuser and the tissue. This in turn reduces surface temperatures and subsequent tissue charring, and reduces the chances of collateral injury. The device's irrigation design provides constant low flow when the therapy is not being applied and a higher flow rate during ablation. The continuous low flow rate irrigation is included to prevent blood, biological fluids or other fluids entering the device's irrigation holes, yet prevents the waste and inconvenience of continuous high flow irrigation. When an ablation is begun the system automatically switches to a flow rate of sufficient magnitude for irrigation. The irrigation system design includes a “loop” in the supply line to provide low flow irrigation.

The device is designed so that it may be labeled as class 1 even though it is driven by 60 W of laser power. This is a great advantage for the surgical and OR staff since it relieves them of the complications of class 4 devices such as protective eyewear, warning lights on the OR door, and entry door interlocks. The class 1 labeling is achievable in part because of the diffused light delivery of the device, and also because of the product's TSS. To make the TSS workable, the E360 includes special coverings on the glass fiber. These coverings act to ensure that the laser system shuts down quickly in the case of a fiber break. The fiber is covered from the laser connector to the handle with a woven stainless steel mesh and two layers of polymer tubing. From within the handle to a point near the diffuser, the fiber is covered by two layers of polymer tubing.

Preferred energy sources for use with the hand held cardiac ablation instrument 10 and the balloon catheter 150 of the present invention include laser light in the range between about 200 nanometers and 2.5 micrometers. In particular, wavelengths that correspond to, or are near, water absorption peaks are often preferred. Such wavelengths include those between about 805 nm and about 1060 nm, preferably between about 900 nm and 1000 nm, most preferably, between about 915 nm and 980 nm. In a preferred embodiment, wavelengths around 915 nm are used during epicardial procedures, and wavelengths around 980 nm are used during endocardial procedures. Suitable lasers include excimer lasers, gas lasers, solid state lasers and laser diodes. One preferred AlGaAs diode array, manufactured by Optopower, Tucson, Ariz., produces a wavelength of 980 nm. Typically the light diffusing element emits between about 2 to about 10 watts/cm of length, preferably between about 3 to about 6 watts/cm, most preferably about 4 watts/cm.

The term “penetrating energy” as used herein is intended to encompass energy sources that do not rely primarily on conductive or convective heat transfer. Such sources include, but are not limited to, acoustic and electromagnetic radiation sources and, more specifically, include microwave, x-ray, gamma-ray, and radiant light sources.

The term “curvilinear,” including derivatives thereof, is herein intended to mean a path or line which forms an outer border or perimeter that either partially or completely surrounds a region of tissue, or separate one region of tissue from another. Further, a “circumferential” path or element may include one or more of several shapes, and may be for example, circular, annular, oblong, ovular, elliptical, or toroidal. The term “clasp” is intended to encompass various types of fastening mechanisms including sutures and magnetic connectors as well as mechanical devices. The term “light” is intended to encompass radiant energy, whether or not visible, including ultraviolet, visible and infrared radiation.

The term “lumen,” including derivatives thereof, is herein intended to mean any elongate cavity or passageway.

The term “transparent” is well recognized in the art and is intended to include those materials which allow transmission of energy. Preferred transparent materials do not significantly impede (e.g., result in losses of over 20 percent of energy transmitted) the energy being transferred from an energy emitter to the tissue or cell site. Suitable transparent materials include fluoropolymers, for example, fluorinated ethylene propylene (FEP), perfluoroalkoxy resin (PFA), polytetrafluoroethylene (PTFE), and ethylene-tetrafluoroethylene (ETFE).

The term “catheter” as used herein is intended to encompass any hollow instrument capable of penetrating body tissue or interstitial cavities and providing a conduit for selectively injecting a solution or gas, including without limitation, venous and arterial conduits of various sizes and shapes, bronchioscopes, endoscopes, cystoscopes, culpascopes, colonscopes, trocars, laparoscopes and the like. Catheters of the present invention can be constructed with biocompatible materials known to those skilled in the art such as those listed supra, e.g., silastic, polyethylene, Teflon, polyurethanes, etc.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. An ablation device for remotely applying ablative energy to biological tissue comprising:
a flexible elongate member having a proximal end, a distal end, and a longitudinal lumen extending therebetween; and
an energy emitting element disposed within the longitudinal lumen of the flexible elongate member and having a proximal end and a distal end for emitting energy along at least a portion of a length of the energy emitting element;
wherein the device is configured to emit a variable amount of energy along a length of the flexible elongate member.

2. The device of claim 1, wherein the energy emitting element comprises a plurality of segments, each segment having a different diameter than an adjacent segment.

3. The device of claim 1, wherein the energy emitting element is tapered along the length thereof.

4. The device of claim 1, further including a reflector for selectively blocking a portion of the energy emitted from the energy emitting element.

5. The device of claim 3, wherein the reflector is disposed around the energy emitting element.

6. The device of claim 5, wherein the reflector includes a window adapted to allow energy to be emitted therethrough.

7. The device of claim 6, wherein the window is adjustable along a length of the reflector.

8. The device of claim 6, wherein a size of the window is adjustable.

9. The device of claim 4, wherein the reflector is formed from a reflective material.

10. The device of claim 7, wherein the reflective material is gold.

11. The device of claim 1, wherein at least a portion of the flexible elongate member is transparent to emitted energy.

12. The ablation device of claim 1, further comprising an elongate balloon disposed within the longitudinal lumen of the flexible elongate member and configured to contact the energy emitting element upon inflation.

13. The ablation device of claim 12, further comprising a controller in communication with the elongate balloon for selectively inflating at least a portion of the elongate balloon.

14. The ablation device of claim 12, wherein inflation of the elongate balloon is adapted to effect an angular orientation of the energy emitting element with respect to the flexible elongate member.

15. The ablation device of claim 12, wherein at least a portion of the flexible elongate member is transparent to emitted energy.

16. The ablation device of claim 1, further comprising a plurality of energy emitting elements, each element having a proximal end and a distal end for emitting energy along a length of the element, and wherein each element has a different length than the other elements.

17. The ablation device of claim 16, further comprising a selection mechanism for selecting at least one of the plurality of energy emitting elements for activation.

18. The device of claim 16, wherein each energy emitting element extends into a diffuser tip at a distal end.

19. The device of claim 16, wherein at least a portion of the flexible elongate member is transparent to emitted energy.
